SEQUENCE LISTING (94 sequences)

SEQ ID NO: 1 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 2 - protein, 495 aa, synthetic construct
KFTIVFPHNQKGNWKNVPSNYHYCPSSSDLNWHNDLIGTAXQVKMPXSHKAIQADGWMCHASKWVTTCDFRWYGPKYITXSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEXELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 3 - protein, 495 aa, synthetic construct
KFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 4 - protein, 495 aa, synthetic construct
KFSIVFPQSQKGNWKNVPSSYHYCPSSSDQNWHNDLLGITMKVKMPXTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEXELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 5 - protein, 497 aa, synthetic construct
QIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGR

SEQ ID NO: 6 - protein, 597 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGGDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMVGMGVSCTVTREDGTN

SEQ ID NO: 7 - protein, 597 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPALFNVPIKEAGGDCHAPTYLPAEVDGDVKLSSNLVILPGQDLQYVLATYDTSAVEHAVVYYVYSPSRLSSYFYPFRLPIKGVPIELQVECFTWDQKLWCRHFCVLADSESGGHITHSGMVGMGVSCTVTREDGTN

SEQ ID NO: 8 - protein, 497 aa, synthetic construct
LHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 9 - protein, 569 aa, synthetic construct
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 10 - protein, 569 aa, synthetic construct
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 11 - protein, 243 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSS

SEQ ID NO: 12 - protein, 240 aa, synthetic construct
DIVMTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKGGGGSGGGGSGGGGSQVQLLESGAELARPGASVKMSCKASGYTFTRYTMHWVKQRPGQGLEWIGYINPSRGYTNYNQKFKDKATLTTDKSSSTAYMQLSSLTSEDSAVYYCAGYYDDHYCLDYWGQGTLVTVSS

SEQ ID NO: 13 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 14 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 15 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 16 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 17 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 18 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 19 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 20 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 21 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 22 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 23 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 24 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 25 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 26 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 27 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 28 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 29 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 30 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 31 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 32 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 33 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 34 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 35 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 36 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 37 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 38 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 39 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 40 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 41 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 42 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 43 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 44 - protein, 495 aa, synthetic construct; identical to SEQ ID NO: 40

SEQ ID NO: 45 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 46 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 47 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 48 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 49 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 50 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 51 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 52 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 53 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 54 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 55 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 56 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 57 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 58 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 59 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 60 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 61 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 62 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 63 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 64 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 65 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 66 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 67 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 68 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 69 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 70 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 71 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 72 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 73 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 74 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 75 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 76 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 77 - protein, 495 aa, synthetic construct
KFSIVFPQSQKGNWKNVPSSYHYCPSSSDQNWHNDLLGITMKVKMPATHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEAELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 78 - protein, 495 aa, synthetic construct
KFSIVFPQSQKGNWKNVPSSYHYCPSSSDQNWHNDLLGITMKVKMPATHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEGELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 79 - protein, 495 aa, synthetic construct
KFSIVFPQSQKGNWKNVPSSYHYCPSSSDQNWHNDLLGITMKVKMPATHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEFELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 80 - protein, 495 aa, synthetic construct
KFSIVFPQSQKGNWKNVPSSYHYCPSSSDQNWHNDLLGITMKVKMPATHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEQELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 81 - protein, 495 aa, synthetic construct
KFSIVFPQSQKGNWKNVPSSYHYCPSSSDQNWHNDLLGITMKVKMPGTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEAELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 82 - protein, 495 aa, synthetic construct
KFSIVFPQSQKGNWKNVPSSYHYCPSSSDQNWHNDLLGITMKVKMPGTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEGELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 83 - protein, 495 aa, synthetic construct
KFSIVFPQSQKGNWKNVPSSYHYCPSSSDQNWHNDLLGITMKVKMPGTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEFELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 84 - protein, 495 aa, synthetic construct
KFSIVFPQSQKGNWKNVPSSYHYCPSSSDQNWHNDLLGITMKVKMPGTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEQELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 85 - protein, 495 aa, synthetic construct
KFSIVFPQSQKGNWKNVPSSYHYCPSSSDQNWHNDLLGITMKVKMPFTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEAELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 86 - protein, 495 aa, synthetic construct
KFSIVFPQSQKGNWKNVPSSYHYCPSSSDQNWHNDLLGITMKVKMPFTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEGELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 87 - protein, 495 aa, synthetic construct
KFSIVFPQSQKGNWKNVPSSYHYCPSSSDQNWHNDLLGITMKVKMPFTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEFELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 88 - protein, 495 aa, synthetic construct
KFSIVFPQSQKGNWKNVPSSYHYCPSSSDQNWHNDLLGITMKVKMPFTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEQELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 89 - protein, 495 aa, synthetic construct
KFSIVFPQSQKGNWKNVPSSYHYCPSSSDQNWHNDLLGITMKVKMPQTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEAELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 90 - protein, 495 aa, synthetic construct
KFSIVFPQSQKGNWKNVPSSYHYCPSSSDQNWHNDLLGITMKVKMPQTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEGELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 91 - protein, 495 aa, synthetic construct
KFSIVFPQSQKGNWKNVPSSYHYCPSSSDQNWHNDLLGITMKVKMPQTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEFELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 92 - protein, 495 aa, synthetic construct
KFSIVFPQSQKGNWKNVPSSYHYCPSSSDQNWHNDLLGITMKVKMPQTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEQELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 93 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 94 - protein, 89 aa, synthetic construct
EMWHEGLEEASRLYFGERNVKGMFEVLEPLHAMMERGPQTLKETSFNQAYGRDLMEAQEWCRKYMKSGNVKDLLQAWDLYYHVFRRISK

(19) United States
(12) Patent Application Publication
(10) Pub. No.: US 2026/0218136 A1
(43) Pub. Date: Jul. 30, 2026

(54) RECOMBINANT RETROVIRUS, COMPOSITIONS, AND METHODS OF USE

(71) Applicant: Kelonia Therapeutics, Inc., Boston, MA (US)

(72) Inventors: Kevin M. Friedman, Boston, MA (US); Michael J. McDonald, Boston, MA (US)

(21) Appl. No.: 19/143,493

(22) PCT Filed: Dec. 29, 2023
(86) PCT No.: PCT/US2023/086463

§ 371 Date: Jun. 26, 2025

Related U.S. Application Data
(60) Provisional application No. 63/477,815, filed on Dec. 29, 2022.
(60) Provisional application No. 63/453,002, filed on Mar. 17, 2023.

(51) Int. Cl. C12N 7/02; A61K 35/76; C07K 14/705; C07K 14/715; C07K 16/28; C12N 5/0781; C12N 5/0783; C12N 15/11; C12N 15/867

(52) U.S. Cl. CPC C12N 7/025; A61K 35/76; C07K 14/70503; C07K 14/7155; C07K 16/2809; C12N 5/0635; C12N 5/0636; C12N 5/0646; C12N 15/11; C12N 15/867; C07K 2319/02; C07K 2319/03; C12N 2740/15021; C12N 2740/15022; C12N 2740/15032; C12N 2740/15043; C12N 2740/15052; C12N 2760/18222; C12N 2760/18422; C12N 2760/20222; C12N 2830/34

(57) ABSTRACT

The present disclosure provides compositions and methods for transducing immune effector cells in vivo with a recombinant retrovirus comprising a polynucleotide encoding a chimeric antigen receptor and a multipartite cell surface receptor.

### CROSS REFERENCE TO RELATED APPLICATIONS

This application claims the benefit under 35 U.S. C. § 119 (e) of U.S. Provisional Application No. 63/477,815, filed Dec. 29, 2022, and U.S. Provisional Application No. 63/453,002, filed Mar. 17, 2023, each of which is incorporated by reference herein in its entirety.

### STATEMENT REGARDING SEQUENCE LISTING

The Sequence Listing associated with this application is provided in text format in lieu of a paper copy and is hereby incorporated by reference into the specification. The name of the text file containing the Sequence Listing is KELO-403-WO1_ST26.xml. The text file is 125 KB, was created on Dec. 12, 2023, and is being submitted electronically via EFS-Web, concurrent with the filing of the specification.

### BACKGROUND

### Technical Field

The present disclosure relates to recombinant retroviruses or retroviral particles for generating modified immune effector cells in vivo to treat cancer and/or B cell malignancies. More particularly, the disclosure relates to recombinant retroviruses or retroviral particles for modifying immune effector cells to express anti-CD19 or anti BCMA chimeric antigen receptors to treat cancer and/or B cell malignancies.

### Description of the Related Art

Patients with aggressive B cell malignancies often undergo a number of standard therapies. Patients that have these failed standard therapies, may have the option to receive an autologous, ex vivo manufactured CAR T cell product that redirects their T cells against B cell antigens. Ex vivo manufacturing of autologous CAR T cell therapies requires a complex and expensive series of steps, which contribute to the expensive price tags associated with such therapies. Allogenic strategies would theoretically alleviate some of this burden but have failed to yield durable treatments in the clinic and are plagued by graft vs. host disease. There exists an unmet need for safer, more cost-effective, and more widely deployable cellular immunotherapies.

### BRIEF SUMMARY

The present disclosure generally relates, in part, to recombinant viruses or retroviral particles comprising a recombinant viral vector comprising a promoter operably linked to a polynucleotide encoding an anti-CD19 or anti-BCMA chimeric antigen receptor and a polynucleotide encoding a modular cell surface receptor. As used herein, the terms “recombinant retrovirus,” “recombinant retroviral particle” and “retroviral particle” are used synonymously and refer to an enveloped retrovirus comprising an engineered retroviral vector. In particular embodiments, the modular cell surface receptor is a chemically inducible cell surface receptor, optionally a rapamycin-activated cell-surface receptor. The modular cell surface receptor comprises first T cell activating protein and a second T cell activating protein. In particular embodiments, the first T cell activating protein comprises an FKBP-rapamycin complex binding protein (FRB) domain or a functional variant thereof and the second T cell activating protein comprises a FK506 binding protein (FKBP) domain or a functional variant thereof; or the first T cell activating protein comprises the FKBP domain or a functional variant thereof and the second T cell activating protein comprises the FRB domain or a functional variant thereof. In particular embodiments, the first T cell activating protein comprises an IL-2Rβ transmembrane and intracellular signaling domain and the second T cell activating protein comprises an IL-2Rγ transmembrane and intracellular signaling domain; or the first T cell activating protein comprises IL-2Rγ transmembrane and intracellular signaling domain and the second T cell activating protein comprises an IL-2Rβ transmembrane and intracellular signaling domain. The recombinant retrovirus or retroviral particle further comprises a viral envelope. The viral envelope comprises a mutated viral envelope glycoprotein that retains fusogenic activity and lacks cognate receptor binding activity (i.e., the ability to bind its cognate receptor on a cell), one or more non-viral membrane-bound tropism polypeptides, and optionally one or more transduction enhancing molecules.

In another aspect, the disclosure provides compositions, pharmaceutical compositions, and kits comprising the recombinant retroviruses contemplated herein.

Methods of treating a disease or disorder in a subject in need thereof comprising administering the recombinant retroviruses, compositions, pharmaceutical compositions, and/or kits contemplated herein are also provided in particular aspects. In particular embodiments, a method of treating a disease or disorder associated with malignant CD19⁺ or BCMA⁺ B cell comprises comprising administering the recombinant retroviruses, compositions, pharmaceutical compositions, and/or kits contemplated herein.

In various embodiments, a recombinant retrovirus comprises (a) a viral envelope comprising (i) one or more mutated viral envelope glycoproteins that retain fusogenic activity and lack cognate receptor binding activity and (ii) a non-viral membrane-bound tropism polypeptide, and (b) a recombinant retroviral vector comprising a polynucleotide encoding a promoter operably linked to a polynucleotide encoding a chimeric antigen receptor (CAR) that binds CD19, CD20, CD22, CD3δ, CD79a, CD79b, GPCR5D, or BCMA and a polynucleotide encoding a modular cell surface receptor; wherein the recombinant retrovirus transduces an immune effector cell in vivo.

In particular embodiments, the recombinant retrovirus is a recombinant lentivirus.

In some embodiments, the recombinant retrovirus is a recombinant lentivirus selected from the group consisting of: human immunodeficiency virus 1 (HIV-1); human immunodeficiency virus 2 (HIV-2), visna-maedi virus (VMV); caprine arthritis-encephalitis virus (CAEV); equine infectious anemia virus (EIAV); feline immunodeficiency virus (FIV); bovine immune deficiency virus (BIV); and simian immunodeficiency virus (SIV).

In certain embodiments, the immune effector cell is selected from the group consisting of: a B lymphocyte, a T lymphocyte, a natural killer (NK) cell, and a natural killer T (NKT) cell.

In further embodiments, the T lymphocyte is an αβ T lymphocyte or a γδ T cell.

In particular embodiments, the one or more mutated viral envelope glycoproteins comprise a vesiculovirus envelope glycoprotein, one or more morbillivirus envelope glycoproteins or one or more henipavirus envelope glycoproteins.

In additional embodiments, the vesiculovirus is selected from the group consisting of: vesicular stomatitis Alagoas virus (VSAV; Alagoas vesiculovirus), Carajás virus (CJSV; Carajas vesiculovirus), Chandipura virus (CHPV; Chandipura vesiculovirus), Cocal virus (COCV; Cocal vesiculovirus), vesicular stomatitis Indiana virus (VSIV; Indiana vesiculovirus), Isfahan virus (ISFV; Isfahan vesiculovirus), Maraba virus (MARAV; Maraba vesiculovirus), Morreton virus (MORV; Morreton vesiculovirus), vesicular stomatitis New Jersey virus (VSNJV; New Jersey vesiculovirus), and Piry virus (PIRYV; Piry vesiculovirus).

In particular embodiments, the vesiculovirus envelope glycoprotein is a vesiculovirus G protein.

In certain embodiments, the vesiculovirus G protein is a COCV G glycoprotein (COCV-G) or a VSIV G glycoprotein (VSIV-G).

In particular embodiments, the VSIV-G envelope protein comprises one or more of: (a) one or more amino acid substitutions at H8, N9, Q10, K47, K50, A51, S183, S179, N180, 1182, M184, Y209, 1347, T350, T352, E353, and R354; (b) an insertion of TT between N9 and Q10, an insertion of GGS between H8 and N9, an insertion of GGS between N9 and Q10, an insertion of TT between N208 and Y209, an insertion of GGS between P46 and K47, and an insertion of GGS between N208 and Y209; or (c) amino acid substitutions at K47 and/or R354; or (d) a deletion of residues 1-8.

In some embodiments, the VSIV-G envelope protein comprises one or more amino acid substitutions at H8, K47, Y209, and R354.

In further embodiments, the VSIV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q.

In particular embodiments, the VSIV-G envelope protein comprises the amino acid sequence set forth in SEQ ID NO: 2, wherein X₁=I, X₂=A, X₃=Q, and X₄=A; X₁=I, X₂=A, X₃=Q, and X₄=G; X₁=I, X₂=A, X₃=Q, and X₄=F; X₁=I, X₂=A, X₃=Q, and X₄=Q; X₁=L, X₂=A, X₃=Q, and X₄=A; X₁=L, X₂=A, X₃=Q, and X₄=G; X₁=L, X₂=A, X₃=Q, and X₄=F; X₁=L, X₂=A, X₃=Q, and X₄=Q; X₁=I, X₂=A, X₃=H, and X₄=A; X₁=I, X₂=A, X₃=H, and X₄=G; X₁=I, X₂=A, X₃=H, and X₄=F; X₁=I, X₂=A, X₃=H, and X₄=Q; X₁=L, X₂=A, X₃=H, and X₄=A; X₁=L, X₂=A, X₃=H, and X₄=G; X₁=L, X₂=A, X₃=H, and X₄=F; X₁=L, X₂=A, X₃=H, and X₄=Q; X₁=I, X₂=G, X₃=Q, and X₄=A; X₁=I, X₂=G, X₃=Q, and X₄=G; X₁=I, X₂=G, X₃=Q, and X₄=F; X₁=I, X₂=G, X₃=Q, and X₄=Q; X₁=L, X₂=G, X₃=Q, and X₄=A; X₁=L, X₂=G, X₃=Q, and X₄=G; X₁=L, X₂=G, X₃=Q, and X₄=F; X₁=L, X₂=G, X₃=Q, and X₄=Q; X₁=I, X₂=G, X₃=H, and X₄=A; X₁=I, X₂=G, X₃=H, and X₄=G; X₁=I, X₂=G, X₃=H, and X₄=F; X₁=I, X₂=G, X₃=H, and X₄=Q; X₁=L, X₂=G, X₃=H, and X₄=A; X₁=L, X₂=G, X₃=H, and X₄=G; X₁=L, X₂=G, X₃=H, and X₄=F; X₁=L, X₂=G, X₃=H, and X₄=Q; X₁=I, X₂=F, X₃=Q, and X₄=A; X₁=I, X₂=F, X₃=Q, and X₄=G; X₁=I, X₂=F, X₃=Q, and X₄=F; X₁=I, X₂=F, X₃=Q, and X₄=Q; X₁=L, X₂=F, X₃=Q, and X₄=A; X₁=L, X₂=F, X₃=Q, and X₄=G; X₁=L, X₂=F, X₃=Q, and X₄=F; X₁=L, X₂=F, X₃=Q, and X₄=Q; X₁=I, X₂=F, X₃=H, and X₄=A; X₁=I, X₂=F, X₃=H, and X₄=G; X₁=I, X₂=F, X₃=H, and X₄=F; X₁=I, X₂=F, X₃=H, and X₄=Q; X₁=L, X₂=F, X₃=H, and X₄=A; X₁=L, X₂=F, X₃=H, and X₄=G; X₁=L, X₂=F, X₃=H, and X₄=F; X₁=L, X₂=F, X₃=H, and X₄=Q; X₁=I, X₂=Q, X₃=Q, and X₄=A; X₁=I, X₂=Q, X₃=Q, and X₄=G; X₁=I, X₂=Q, X₃=Q, and X₄=F; X₁=I, X₂=Q, X₃=Q, and X₄=Q; X₁=L, X₂=Q, X₃=Q, and X₄=A; X₁=L, X₂=Q, X₃=Q, and X₄=G; X₁=L, X₂=Q, X₃=Q, and X₄=F; X₁=L, X₂=Q, X₃=Q, and X₄=Q; X₁=I, X₂=Q, X₃=H, and X₄=A; X₁=I, X₂=Q, X₃=H, and X₄=G; X₁=I, X₂=Q, X₃=H, and X₄=F; X₁=I, X₂=Q, X₃=H, and X₄=Q; X₁=L, X₂=Q, X₃=H, and X₄=A; X₁=L, X₂=Q, X₃=H, and X₄=G; X₁=L, X₂=Q, X₃=H, and X₄=F; and X₁=L, X₂=Q, X₃=H, and X₄=Q.

In additional embodiments, the vesiculovirus G protein is COCV-G.

In certain embodiments, the COCV-G envelope protein comprises one or more amino acid substitutions at K47 and R354.

In some embodiments, the COCV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q.

In particular embodiments, the COCV-G envelope protein comprises the amino acid sequence set forth in SEQ ID NO: 4, wherein X₁=A and X₂=A; X₁=A and X₂=G; X₁=A and X₂=F; X₁=A and X₂=Q; X₁=G and X₂=A; X₁=G and X₂=G; X₁=G and X₂=F; X₁=G and X₂=Q; X₁=F and X₂=A; X₁=F and X₂=G; X₁=F and X₂=F; X₁=F and X₂=Q; X₁=Q and X₂=A; X₁=Q and X₂=G; X₁=Q and X₂=F; or X₁=A and X₂=Q.

In additional embodiments, the one or more morbillivirus envelope glycoproteins are measles virus F (MV-F) and measles virus H (MV-H).

In further embodiments, the MV-H protein comprises one or more amino acid substitutions at Y481, R533, S548, and F549.

In further embodiments, the MV-H protein comprises one or more amino acid substitutions selected from the group consisting of: Y481A, R533A, S548L, and F549S.

In particular embodiments, the one or more henipavirus envelope glycoproteins are nipah virus F (NiV-F) and nipah virus G (NiV-G).

In particular embodiments, the NiV-G protein comprises one or more amino acid substitutions at E501, W504, Q530, and E533.

In certain embodiments, the NiV-G protein comprises one or more amino acid substitutions at E501A, W504A, Q530A, and E533A.

In further embodiments, the non-viral membrane-bound tropism polypeptide comprises an extracellular antigen targeting domain, a spacer polypeptide, and a transmembrane domain.

In particular embodiments, the extracellular antigen targeting domain binds an antigen expressed on an immune effector cell.

In some embodiments, the extracellular antigen targeting domain binds an antigen expressed on an immune effector cell selected from the group consisting of: the alpha, beta, gamma, or delta chain of the T cell receptor, CD2, CD3δ, CD3ε CD3γ, CD4, CD8α, and CD8β.

In some embodiments, the extracellular antigen targeting domain comprises an antibody or antigen binding fragment thereof that binds an antigen expressed on the immune effector cell.

In particular embodiments, the extracellular antigen targeting domain comprises an anti-CD3 antibody or antigen binding fragment selected from the group consisting of OKT3, UCHT1, YTH12.5, TR66, and variants thereof, e.g., teplizumab, and antibodies and antigen binding fragments that have at least 80%, at least 85%, at least 90%, at least 95%, at least 99%, or 100% identity thereto.

In additional embodiments, the promoter is an inducible promoter or an MNDU3 promoter.

In further embodiments, the polynucleotide encoding the chimeric antigen receptor is 5′ to the polynucleotide encoding the modular cell surface receptor.

In additional embodiments, the polynucleotide encoding the chimeric antigen receptor is 3′ to the polynucleotide encoding the modular cell surface receptor.

In certain embodiments, the modular cell surface receptor is a chemically inducible cell surface receptor.

In particular embodiments, the multipartite cell-surface receptor is a rapamycin-activated cell-surface receptor.

In certain embodiments, the modular cell surface receptor comprises a first T cell activating protein and a second T cell activating protein.

In further embodiments, the first T cell activating protein comprises an FKBP-rapamycin complex binding protein (FRB) domain or a functional variant thereof and the second T cell activating protein comprises a FK506 binding protein (FKBP) domain or a functional variant thereof; or the first T cell activating protein comprises the FKBP domain or a functional variant thereof and the second T cell activating protein comprises the FRB domain or a functional variant thereof.

In some embodiments, the first T cell activating protein comprises an IL-2Rβ transmembrane and intracellular signaling domain and the second T cell activating protein comprises an IL-2Rγ transmembrane and intracellular signaling domain; or the first T cell activating protein comprises IL-2Rγ transmembrane and intracellular signaling domain and the second T cell activating protein comprises an IL-2Rβ transmembrane and intracellular signaling domain.

In particular embodiments, the recombinant retroviral vector comprises a polynucleotide encodes a polypeptide that confers resistance to an immunosuppressive agent.

In additional embodiments, the polypeptide that confers resistance to an immunosuppressive agent binds rapamycin, wherein optionally, the polypeptide comprises an FRB domain or a functional variant thereof.

In certain embodiments, the anti-CD19 chimeric antigen receptor comprises an antigen binding domain comprising an amino acid sequence set forth in SEQ ID NO: 93 or an antigen binding domain having at least 95% identity thereto.

In further embodiments, the recombinant retroviral vector comprises a polynucleotide encoding a dominant negative TGFβ receptor or a TGFβ signal convertor.

In additional embodiments, expression of the chimeric antigen receptor is modulated by an FRB-degron fusion polypeptide and wherein suppression of the FRB-degron fusion polypeptide is chemically inducible by a ligand, optionally, wherein the ligand is rapamycin or a rapalog.

In certain embodiments, the viral envelope further comprises one or more transduction enhancers, wherein the transduction enhancer is selected from the group consisting of a T cell activation receptor, a NK-cell activation receptor, and a co-stimulatory molecule.

In some embodiments, the one or more transduction enhancers comprise one or more of CD80, CD86, CD137L, OX40L, and ICOSL.

In additional embodiments, a pharmaceutical composition comprises a recombinant retrovirus contemplated herein and a pharmaceutically acceptable diluent.

In particular embodiments, a kit comprises a pharmaceutical composition contemplated herein and optionally a composition comprising a ligand, optionally wherein the ligand is rapamycin.

In certain embodiments, a method of treating a disease or disorder in a subject in need thereof, comprises administering to a subject, a therapeutically effective amount of a recombinant retrovirus or pharmaceutical composition contemplated herein.

In some embodiments, a method of treating a disease or disorder associated with malignant CD19+ or BCMA+ B cells comprises administering to a subject, a recombinant retrovirus or pharmaceutical composition contemplated herein.

In particular embodiments, the recombinant retrovirus is administered by intraperitoneal, subcutaneous, or intranodal injection.

In further embodiments, the recombinant retrovirus is administered by intranodal injection, via inguinal lymph node.

In particular embodiments, the disease or disorder comprises B cell malignancy, relapsed/refractory CD19-expressing malignancy, diffuse large B cell lymphoma (DLBCL), Burkitt's type large B cell lymphoma (B-LBL), follicular lymphoma (FL), chronic lymphocytic leukemia (CLL), acute lymphocytic leukemia (ALL), mantle cell lymphoma (MCL), hematological malignancy, colon cancer, lung cancer, liver cancer, breast cancer, renal cancer, prostate cancer, ovarian cancer, skin cancer, melanoma, bone cancer, brain cancer, squamous cell carcinoma, leukemia, myeloma, B cell lymphoma, kidney cancer, uterine cancer, adenocarcinoma, pancreatic cancer, chronic myelogenous leukemia, glioblastoma, neuroblastoma, medulloblastoma, sarcoma, and any combination thereof.

In some embodiments, the disease or disorder comprises diffuse large B cell lymphoma (DLBCL).

In certain embodiments, at least 2 million, at least 4 million, at least 6 million, at least 8 million or at least 10 million or more transducing units of recombinant retrovirus are administered to the subject.

In particular embodiments, a method of transducing an immune effector cell in vivo, comprises administering to a subject, a recombinant retrovirus or a pharmaceutical composition contemplated herein.

In various embodiments, a method of making a recombinant retrovirus comprises: a) transfecting the host cell with one or more polynucleotides that express retroviral gag-pol, one or more mutated viral envelope glycoproteins that retain fusogenic activity and lack cognate receptor binding activity, a non-viral membrane-bound tropism polypeptide, and optionally rev and a transfer vector encoding a recombinant retroviral vector contemplated herein; and b) culturing the transduced cell for about 1 to 3 days to produce the recombinant retrovirus.

Further aspects and embodiments of the disclosure are provided by the following detailed description.

### BRIEF DESCRIPTION OF THE SEQUENCE IDENTIFIERS

SEQ ID NOs: 1-10 set forth amino acid sequences of fusogens.

SEQ ID NOs: 11-12 set forth amino acid sequences of anti-CD3 antibodies.

SEQ ID NOs: 13-92 set forth amino acid sequences of fusogens.

SEQ ID NO: 93 sets forth an amino acid sequence of an anti-CD19 antibody.

SEQ ID NO: 94 sets forth an amino acid sequence of an FRB protein.

In the foregoing sequences, X, if present, refers to any amino acid, a specified group of amino acids or the absence of an amino acid.

Throughout the disclosure, the amino acid position(s) of a fusogen is with reference to the fusogen lacking a signal sequence (i.e., the amino acid sequence after the signal peptide has been cleaved).

### DETAILED DESCRIPTION

### a. Overview

The disclosure generally relates to a recombinant retrovirus comprising a viral envelope comprising a mutated viral envelope glycoprotein modified to retain fusogenic activity and decrease, reduce, substantially ablate, ablate, abolish or eliminate cell binding or attachment activity, one or more non-viral membrane-bound tropism polypeptides, and a viral vector comprising a polynucleotide sequence encoding an anti-CD19 or anti-BCMA chimeric antigen receptor and a modular cell surface receptor, wherein the recombinant retrovirus transduces immune cells in vivo. It is contemplated that a recombinant retrovirus transduces T cells in vivo to express an anti-CD19 or anti-BCMA CAR and target CD19- or BCMA-expressing tumor cells. In particular embodiments, a recombinant retrovirus has a multi-step mechanism of action: the recombinant retrovirus binds to T cells in vivo via one or more non-viral membrane-bound tropism polypeptides comprising an anti-CD3 scFv, activates the T cells and facilitates recombinant retrovirus internalization through interaction with the mutated viral envelope glycoprotein that retains fusogenic activity and lacks cognate receptor binding activity (i.e., modified to retain fusogenic activity and decrease, reduce, substantially ablate, ablate, abolish or eliminate cell binding or attachment activity); the retroviral vector encoding an anti-CD19 or anti-BCMA CAR and a modular cell surface receptor comprising an FRB domain is reverse-transcribed into DNA, shuttled to the nucleus, and integrated into the genome; and the transduced T cells express the CAR and bind to target cells expressing CD19 or BCMA, while also expressing the FRB comprising modular surface receptor for rapamycin-controlled cytokine signaling.

The methods and compositions subjects herein may facilitate administering the recombinant retroviral particle directly into the subjects in need of treatment.

The methods and compositions contemplated herein to activate and transduce non-stimulated T cells, which then express the anti-CD19 CAR or anti-BCMA CAR, respond to rapamycin treatment with enhanced proliferation, and kill CD19⁺ or BCMA⁺ malignant B cells.

The present disclosure provides a method of treating a disease or disorder, transducing immune cells in vivo, and/or generating an immune cell expressing an anti-CD19 CAR or anti-BCMA CAR in a subject in need thereof, comprising administering a recombinant retroviral particle contemplated herein to the subject. In particular embodiments, the method further comprises administering rapamycin to the subject. In particular embodiments, the methods contemplated herein eliminate the need for pre-activation of the immune cells prior to administration of the viral particle. In particular embodiments, the method comprises no pre-activation of the immune cells in the subject prior to administration of the recombinant retroviral particle. In particular embodiments, the methods contemplated herein do not comprise administering separate CD3 and/or CD28 activating agents prior to administration of the recombinant retroviral particle.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 4th ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience (2002); Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid the Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells and Enzymes (IRL Press, 1986); the treatise, Methods in Enzymology (Academic Press, Inc., N.Y.); Gene Transfer Vectors for Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods in Cell and Molecular Biology (Mayer and Walker, eds., Academic Press, London, 1987); Handbook of Experimental Immunology, Volumes I-IV (D. M. Weir and CC Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Retroviral Particles

In particular embodiments, a recombinant retrovirus or recombinant retroviral particle comprises a vector comprising one or more polynucleotides. In certain embodiments, the one or more polynucleotides encode one or more therapeutic polypeptides. A “therapeutic polypeptide” refers to a polypeptide which is being developed for therapeutic use, or which has been developed for therapeutic use. In particular embodiments, a therapeutic polypeptide is expressed in a target cell (e.g., host T cell) for therapeutic use. In particular embodiments, a therapeutic polypeptide comprises an engineered antigen receptor, e.g., a T cell receptor (TCR), a chimeric antigen receptor (CAR), a dimerization activated receptor initiation complex (DARIC), or a cytokine receptor.

Retroviruses include, without limitation, lentiviruses, gamma-retroviruses, and alpha-retroviruses. Retroviruses contemplated herein are used to deliver polynucleotides to immune effector cells. Lentiviruses are complex retroviruses, which, in addition to the common retroviral genes gag, pol, and env, contain other viral accessory genes. Illustrative retroviruses suitable for use in particular embodiments contemplated herein, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV) and lentivirus.

As used herein, the term “lentivirus” refers to a group (or genus) of complex retroviruses. Illustrative lentiviruses include, but are not limited to, HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV).

Recombinant retroviruses contemplated herein comprise genomes that lack one or more viral accessory genes, e.g., the genes env, vif, vpr, vpu and nef, thereby increasing the safety of the retroviruses. In particular embodiments, a recombinant virus contains two copies of a vector, a genomic RNA comprising backbone sequences derived from a lentivirus. It is understood that many different sources of retroviral and/or lentiviral sequences can be used, or combined and numerous substitutions and alterations in certain of the lentiviral sequences may be accommodated without impairing the ability of a vector to package polynucleotides into a recombinant retrovirus or retroviral particle and to transfer the polynucleotide into a target cell. Illustrative examples of recombinant retroviruses and retroviral vectors include, but are not limited to those described in Naldini et al., (1996a, 1996b, and 1998); Zufferey et al., (1997); Dull et al., 1998, U.S. Pat. Nos. 6,013,516; and 5,994,136, each of which are incorporated herein by reference in its entirety.

In particular embodiments, a lentivirus is manufactured using a third-generation lentiviral vector system comprising an envelope plasmid encoding a viral envelope glycoprotein (e.g., one or more mutated viral envelope glycoproteins that retain fusogenic activity and lack cognate receptor binding activity) and optionally, one or more non-viral membrane-bound tropism polypeptide and/or transduction enhancer polypeptides, two packaging plasmids (e.g., one encoding gag-pol and one encoding rev) and a transfer plasmid or vector. The “transfer plasmid” or “transfer vector” encodes a viral genomic RNA (e.g., vector backbone) comprising the polynucleotide sequence that is delivered by recombinant lentivirus to a target cell. In particular embodiments, a transfer plasmid comprises one or more transgene sequences of interest flanked by long terminal repeat (LTR) sequences, which facilitate packaging, reverse transcription and integration of the viral vector and associated polynucleotide sequences into the host genome. Viral vectors contemplated herein are replication incompetent, i.e., lack the genetic elements necessary for generation of infective particles in the host cell. For example, the viral vector may be designed with a deletion of the 3′ LTR, rendering the virus “self-inactivating” (SIN).

In particular embodiments, a transfer plasmid comprises a polynucleotide sequence encoding a retroviral vector comprising, from 5′ to 3′: an RSV promoter, a 5′ LTR, an HIV-1 packaging signal (Psi), an HIV-1 Rev response element (RRE), a cPPT/CTS, an MNDU3 promoter, a human CSF2R signal peptide, an anti-CD19 scFv, an IgG4 hinge domain, a human CD28 transmembrane domain, a 4-1BB costimulatory domain, a CD3ζ primary signaling domain, a P2A sequence, a cytosolic FRB domain, a P2A sequence, an NGAL signal peptide, an FKBP12 polypeptide, an IL2Rγ transmembrane and cytoplasmic domain, a P2A sequence, a CD8α signal peptide, an FRB T2098L polypeptide, an IL2Rβ transmembrane and cytoplasmic domain, a WPRE, and a 3′ SIN LTR.

In particular embodiments, a transfer plasmid comprises a polynucleotide sequence encoding a retroviral vector comprising, from 5′ to 3′: a human cytomegalovirus (CMV) immediate early enhancer and CMV promoter, a 5′ LTR, an HIV-1 packaging signal (Psi), an HIV-1 Rev response element (RRE), a cPPT/CTS, an MNDU3 promoter, a human CSF2R signal peptide, an anti-CD19 scFv, an IgG4 hinge domain, a human CD28 transmembrane domain, a 4-1BB costimulatory domain, a CD3ζ primary signaling domain, a P2A sequence, a cytosolic FRB domain, a P2A sequence, an NGAL signal peptide, an FKBP12 polypeptide, an IL2Rγ transmembrane and cytoplasmic domain, a P2A sequence, a CD8α signal peptide, an FRB T2098L polypeptide, an IL2Rβ transmembrane and cytoplasmic domain, a 3′ SIN LTR, and a synthetic poly A signal.

In particular embodiments, the envelope plasmid and/or packaging plasmids further encode one or more non-viral membrane-bound tropism polypeptide and/or transduction enhancer polypeptides.

Recombinant retroviruses or retroviral particles are manufactured using a “packaging cell line.” In particular embodiments, recombinant retroviruses or retroviral particles are produced by transfecting a packaging cell line with the transfer plasmid, packaging plasmid(s), and envelope plasmid.

As used herein, the terms “retroviral vector” or “lentiviral vector” refer to a nucleic acid that encodes a retroviral or lentiviral genome comprising one or more polynucleotide sequences to be delivered to a target cell. In particular embodiments, a recombinant retrovirus or recombinant lentivirus comprises a genomic RNA, e.g., a vector, a lipid-bilayer envelope, and other accessory proteins including integrase, protease, and matrix protein. The vectors may further comprise one of more accessory elements to increase transduction efficiency (e.g., a FLAP element that includes the central polypurine tract and central termination sequences (cPPT and CTS)), viral packaging (e.g., a Psi(ψ) packaging signal, a rev response element (RRE)), and/or other elements that increase therapeutic gene expression (e.g., poly(A) sequences), and may optionally comprise a woodchuck post-transcriptional regulatory element (WPRE) or hepatitis B virus post-transcriptional regulatory element (HPRE).

The efficiency of retroviral or lentiviral vector manufacturing may be assessed in various ways, including measurement of vector copy number (VCN) or vector genomes (vg) such as by quantitative polymerase chain reaction (qPCR), or titer of the virus in infectious units per milliliter (IU/mL). For example, the titer may be assessed using a functional assay performed on the cultured tumor cell line HT1080 or on HeLa cells.

In particular embodiments, a recombinant retrovirus contemplated herein comprises a vector encoding a promoter operably linked to a polynucleotide encoding an engineered antigen receptor that specifically binds CD19 or BCMA and optionally a modular cell surface receptor. Illustrative promoters suitable for use in particular embodiments contemplated herein include, but are not limited to, an EFla promoter, a cytomegalovirus (CMV) promoter, a CAG promoter, an SV40 promoter, an SV40/CD43 promoter, and a MNDU3 promoter.

In particular embodiments, a recombinant retrovirus comprises a viral envelope comprising or expressing one or more non-viral membrane-bound tropism polypeptides and/or one or more transduction enhancers. In particular embodiments, a recombinant retrovirus comprises a vector comprising a polynucleotide encoding a modular cell surface receptor. In particular embodiments, a recombinant retrovirus comprises a vector comprising a polynucleotide encoding an engineered antigen receptor. In particular embodiments, a recombinant retrovirus comprises one or more tagging proteins.

In particular embodiments, a recombinant retrovirus comprises a vector comprising a polynucleotide comprising, from 5′ to 3′: a 5′ long terminal repeat (LTR) comprising an R region and U5 region, a polynucleotide encoding a promoter, one or more polynucleotides encoding an engineered antigen receptor and/or a modular cell surface receptor, a 3′ LTR comprising a U3 region and an R region, and optionally a poly(A) tail.

In particular embodiments, a recombinant retrovirus is a recombinant lentivirus comprising: a viral envelope that expresses a mutated viral envelope glycoprotein that retains fusogenic activity and lack cognate receptor binding activity, and a non-viral membrane-bound tropism polypeptide comprising an anti-CD3 scFv; and a SIN retroviral vector comprising an MNDU3 promoter, a transgene encoding an anti-CD19 CAR comprising an FMC63 scFv and 4-1BB and CD32 signaling domains, and/or a modular cell surface receptor comprising an inducible T cell proliferative signaling system (rapamycin-activated cytokine receptor, RACR), and/or an FRB polypeptide derived from the mammalian target of rapamycin (mTOR) complex that binds intracellular rapamycin to confer rapamycin resistance to transduced cells.

### C. Viral Envelope

In particular embodiments, a recombinant retrovirus comprises a viral envelope comprising one or more mutated viral envelope glycoproteins that mediate fusion of the viral particle and the target host cell but that do not bind its cognate receptor expressed on the target host cell and one or more non-viral membrane-bound tropism polypeptides.

In particular embodiments, one or more mutated viral envelope glycoproteins comprise a vesiculovirus envelope glycoprotein or one or more morbillivirus envelope glycoproteins or henipavirus envelope glycoproteins. In particular embodiments, the mutated viral envelope glycoprotein comprises a heterologous signal peptide.

In particular embodiments, one or more mutated viral envelope glycoproteins comprise a vesiculovirus envelope glycoprotein or one or more morbillivirus envelope glycoproteins or henipavirus envelope glycoproteins.

Illustrative examples of vesiculoviruses suitable for use in particular embodiments from which G glycoproteins can be isolated from include, but are not limited to vesicular stomatitis Alagoas virus (VSAV; Alagoas vesiculovirus), Carajás virus (CJSV; Carajas vesiculovirus), Chandipura virus (CHPV; Chandipura vesiculovirus), Cocal virus (COCV; Cocal vesiculovirus), vesicular stomatitis Indiana virus (VSIV, f.k.a. VSV; Indiana vesiculovirus), Isfahan virus (ISFV; Isfahan vesiculovirus), Maraba virus (MARAV; Maraba vesiculovirus), Morreton virus (MORV; Morreton vesiculovirus), vesicular stomatitis New Jersey virus (VSNJV; New Jersey vesiculovirus), and Piry virus (PIRYV; Piry vesiculovirus).

In particular embodiments, a vesiculovirus G envelope protein comprises one or more amino acid substitutions that that enable the polypeptide to mediate fusion of the viral particle and the target host cell but that ablate the polypeptide's ability to bind its cognate receptor expressed on the target host cell, e.g., LDL-R. In particular embodiments, a vesiculovirus G envelope protein comprises an amino acid sequence set forth in any one of SEQ ID NOs: 15-322 disclosed in U.S. patent application No. 20200216502, each said sequence incorporated by reference herein in its entirety, or an amino acid sequence at least 95%, 96%, 97%, 98%, or 99% identical thereto that enables the polypeptide to mediate fusion of the viral particle and the target host cell but that ablate the polypeptide's ability to bind its cognate receptor expressed on the target host cell, e.g., LDL-R. In particular embodiments, a vesiculovirus G envelope protein comprises amino acid substitution at positions 47 and/or 354 (or 358 in some strains, e.g., CJSV, VSNJV) with reference to the vesiculovirus G envelope protein lacking a signal peptide.

In particular embodiments, a vesiculovirus is vesicular stomatitis Indiana virus (VSIV). In particular embodiments, a mutated viral envelope glycoprotein is derived from a VSIV envelope glycoprotein (VSIV-G; e.g., SEQ ID NO: 1: KFTIVFPHNQKGNWKNVPSNYHY CPSSSDLNWHNDLIGTALQVKMPKSHKAIQADGWMCHASKWVTTCDFRWYGP KYITHSIRSFTPSVEQCKESIEQTKQGTWLNPGFPPQSCGYATVTDAEAVIVQVTP HHVLVDEYTGEWVDSQFINGKCSNYICPTVHNSTTWHSDYKVKGLCDSNLISMD ITFFSEDGELSSLGKEGTGFRSNYFAYETGGKACKMQYCKHWGVRLPSGVWFEM ADKDLFAAARFPECPEGSSISAPSQTSVDVSLIQDVERILDYSLCQETWSKIRAGLP ISPVDLSYLAPKNPGTGPAFTIINGTLKYFETRYIRVDIAAPILSRMVGMISGTTTER ELWDDWAPYEDVEIGPNGVLRTSSGYKFPLYMIGHGMLDSDLHLSSKAQVFEHP HIQDAASQLPDDESLFFGDTGLSKNPIELVEGWFSSWKSSIASFFFIIGLIIGLFLVLR VGIHLCIKLKHTKKRQIYTDIEMNRLGK) or an amino acid sequence at least 95%, 96%, 97%, 98%, or 99% identical thereto comprising one or more modifications that enable the polypeptide to mediate fusion of the viral particle and a cell but that substantially ablate or ablate the polypeptide's ability to bind its cognate receptor expressed on a cell, e.g., LDL-R. In particular embodiments, a mutated viral envelope glycoprotein is derived from a VSIV-G polypeptide set forth in SEQ ID NO: 1 comprising L47I and/or H80Q amino acid substitutions, such substitutions being present in naturally occurring variants of VSIV.

In particular embodiments, a mutated VSIV-G envelope protein comprises one or more of: one or more amino acid substitutions at H8, N9, Q10, K47, K50, A51, S183, S179, N180, I182, M184, Y209, 1347, T350, T352, E353, and R³⁵⁴ (substitution with any amino acid; a conservation substitution; a disruptive substitution; substitution with D, E, A, G, F, or Q; or substitution with A, G, F, or Q); an insertion of TT between N9 and Q10, an insertion of GGS between H8 and N9, an insertion of GGS between N9 and Q10, an insertion of TT between N208 and Y209, an insertion of GGS between P46 and K47, and an insertion of GGS between N208 and Y209; or a deletion of residues 1-8. In particular embodiments, a VSIV-G envelope protein comprises one or more amino acid substitutions at H8, K47, Y209, and R354. In particular embodiments, a VSIV-G envelope protein comprises one or more amino acid substitutions at H8, K47, Y209, and R354, wherein each substitution can be with A, G, F, or Q. Amino acid positions are with reference to a VSIV-G envelope protein that lacks a signal peptide, e.g., SEQ ID NO: 1. In particular embodiments, a mutated viral envelope glycoprotein is derived from a VSIV-G polypeptide set forth in SEQ ID NO: 1 comprising L47I and/or H80Q amino acid substitutions, such substitutions present in naturally occurring variants of VSIV.

In particular embodiments, a mutated VSIV-G polypeptide comprises one or more amino acid substitutions at K47, 1182, and/or R354 (substitution with any amino acid; a conservation substitution; a disruptive substitution; substitution with D, E, A, G, F, or Q; or substitution with A, G, F, or Q). In particular embodiments, a mutated VSIV-G polypeptide comprises amino acid substitutions at K47, I182, or R354; K47 and I182; K47 and R354; I182 and R354; or at K47, I182, and R354 of SEQ ID NO: 1.

In particular embodiments, a mutated VSIV-G polypeptide comprises one or more of the following amino acid substitutions: K47A, K47Q, I182E, I182D, R354A, and/or R354Q. In particular embodiments, a mutated VSIV-G polypeptide comprises the following amino acid substitutions: K47A, K47Q, I182E, I182D, R354A, or R354Q; K47A and I182E; K47A and I182D; K47Q and I182E; K47Q and I182D; I182E and R354A; I182E and R354Q; I182D and R354A; I182D and R354Q; K47A and R354A; K47A and R354Q; K47Q and R354A; K47Q and R354Q; K47A, I182E, and R354A; K47A, I182D, and R354A; K47Q, I182E, and R354A; K47Q, I182D, and R354A; K47A, I182E, and R354Q; K47A, I182D, and R354Q; K47Q, I182E, and R354Q; or K47Q, I182D, and R354Q of SEQ ID NO: 1.

In particular embodiments, a VSIV-G envelope protein comprises one or more amino acid substitutions at H8, K47, Y209, and R354. In particular embodiments, a VSIV-G envelope protein comprises one or more amino acid substitutions at H8, K47, Y209, and R354, wherein each substitution can be with A, G, F, or Q. In particular embodiments, a VSIV-G envelope protein comprises one or more amino acid substitutions at K47 and R354 (with reference to a VSIV-G envelope protein that lacks a signal peptide, e.g., SEQ ID NO: 1). In particular embodiments, a VSIV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q. In preferred embodiments, a VSIV-G envelope comprises the amino acid substitutions K47Q or K47A and R354A or R354Q. Amino acid positions are with reference to a VSIV-G envelope protein that lacks a signal peptide.

In particular embodiments, a VSIV-G envelope protein comprises an amino acid sequence set forth in SEQ ID NO: 2 (wherein X₁=I, X₂=A, X₃=Q, and X₄=A; X₁=I, X₂=A, X₃=Q, and X₄=G; X₁=I, X₂=A, X₃=Q, and X₄=F; X₁=I, X₂=A, X₃=Q, and X₄=Q; X₁=L, X₂=A, X₃=Q, and X₄=A; X₁=L, X₂=A, X₃=Q, and X₄=G; X₁=L, X₂=A, X₃=Q, and X₄=F; X₁=L, X₂=A, X₃=Q, and X₄=Q; X₁=I, X₂=A, X₃=H, and X₄=A; X₁=I, X₂=A, X₃=H, and X₄=G; X₁=I, X₂=A, X₃=H, and X₄=F; X₁=I, X₂=A, X₃=H, and X₄=Q; X₁=L, X₂=A, X₃=H, and X₄=A; X₁=L, X₂=A, X₃=H, and X₄=G; X₁=L, X₂=A, X₃=H, and X₄=F; X₁=L, X₂=A, X₃=H, and X₄=Q; X₁=I, X₂=G, X₃=Q, and X₄=A; X₁=I, X₂=G, X₃=Q, and X₄=G; X₁=I, X₂=G, X₃=Q, and X₄=F; X₁=I, X₂=G, X₃=Q, and X₄=Q; X₁=L, X₂=G, X₃=Q, and X₄=A; X₁=L, X₂=G, X₃=Q, and X₄=G; X₁=L, X₂=G, X₃=Q, and X₄=F; X₁=L, X₂=G, X₃=Q, and X₄=Q; X₁=I, X₂=G, X₃=H, and X₄=A; X₁=I, X₂=G, X₃=H, and X₄=G; X₁=I, X₂=G, X₃=H, and X₄=F; X₁=I, X₂=G, X₃=H, and X₄=Q; X₁=L, X₂=G, X₃=H, and X₄=A; X₁=L, X₂=G, X₃=H, and X₄=G; X₁=L, X₂=G, X₃=H, and X₄=F; X₁=L, X₂=G, X₃=H, and X₄=Q; X₁=I, X₂=F, X₃=Q, and X₄=A; X₁=I, X₂=F, X₃=Q, and X₄=G; X₁=I, X₂=F, X₃=Q, and X₄=F; X₁=I, X₂=F, X₃=Q, and X₄=Q; X₁=L, X₂=F, X₃=Q, and X₄=A; X₁=L, X₂=F, X₃=Q, and X₄=G; X₁=L, X₂=F, X₃=Q, and X₄=F; X₁=L, X₂=F, X₃=Q, and X₄=Q; X₁=I, X₂=F, X₃=H, and X₄=A; X₁=I, X₂=F, X₃=H, and X₄=G; X₁=I, X₂=F, X₃=H, and X₄=F; X₁=I, X₂=F, X₃=H, and X₄=Q; X₁=L, X₂=F, X₃=H, and X₄=A; X₁=L, X₂=F, X₃=H, and X₄=G; X₁=L, X₂=F, X₃=H, and X₄=F; X₁=L, X₂=F, X₃=H, and X₄=Q; X₁=I, X₂=Q, X₃=Q, and X₄=A; X₁=I, X₂=Q, X₃=Q, and X₄=G; X₁=I, X₂=Q, X₃=Q, and X₄=F; X₁=I, X₂=Q, X₃=Q, and X₄=Q; X₁=L, X₂=Q, X₃=Q, and X₄=A; X₁=L, X₂=Q, X₃=Q, and X₄=G; X₁=L, X₂=Q, X₃=Q, and X₄=F; X₁=L, X₂=Q, X₃=Q, and X₄=Q; X₁=I, X₂=Q, X₃=H, and X₄=A; X₁=I, X₂=Q, X₃=H, and X₄=G; X₁=I, X₂=Q, X₃=H, and X₄=F; X₁=I, X₂=Q, X₃=H, and X₄=Q; X₁=L, X₂=Q, X₃=H, and X₄=A; X₁=L, X₂=Q, X₃=H, and X₄=G; X₁=L, X₂=Q, X₃=H, and X₄=F; and X₁=L, X₂=Q, X₃=H, and X₄=Q) or an amino acid sequence at least 95%, 96%, 97%, 98%, or 99% identical thereto that mediates fusion of the viral particle and the target host cell but that does not bind its cognate receptor expressed on the target host cell, e.g., LDL-R.

TABLE 1

| SEQ ID | |
|---|---|
| NO | Amino Acid Sequence |
| 2 | KFTIVFPHNQKGNWKNVPSNYHYCPSSSDLNWHNDLIGTAX₁Q |
| | VKMPX₂SHKAIQADGWMCHASKWVTTCDFRWYGPKYITX₃SIRS |
| | FTPSVEQCKESIEQTKQGTWLNPGFPPQSCGYATVTDAEAVIV |
| | QVTPHHVLVDEYTGEWVDSQFINGKCSNYICPTVHNSTTWHSD |
| | YKVKGLCDSNLISMDITFFSEDGELSSLGKEGTGFRSNYFAYE |
| | TGGKACKMQYCKHWGVRLPSGVWFEMADKDLFAAARFPECPEG |
| | SSISAPSQTSVDVSLIQDVERILDYSLCQETWSKIRAGLPISP |
| | VDLSYLAPKNPGTGPAFTIINGTLKYFETRYIRVDIAAPILSR |
| | MVGMISGTTTEX₄ELWDDWAPYEDVEIGPNGVLRTSSGYKFPL |
| | YMIGHGMLDSDLHLSSKAQVFEHPHIQDAASQLPDDESLFFGD |
| | TGLSKNPIELVEGWFSSWKSSIASFFFIIGLIIGLFLVLRVGI |
| | HLCIKLKHTKKRQIYTDIEMNRLGK |

In particular embodiments, a mutated VSIV-G envelope protein comprises an amino acid sequence set forth in any one of SEQ ID NOs: 13-76 or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R. In particular embodiments, a mutated VSIV-G envelope protein comprises an amino acid sequence set forth in any one of SEQ ID NOs: 61-76 or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R. In particular embodiments, a mutated VSIV-G envelope protein comprises an amino acid sequence set forth in any one of SEQ ID NOs: 61, 65, 69, and 73 or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R.

TABLE 2

| SEQ ID | |
|---|---|
| NO: | AMINO ACID SEQUENCE |
| 13 | KFTIVFPHNQKGNWKNVPSNYHYCPSSSDLNWHNDLIGTAIQVKMPASHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 14 | KFTIVFPHNQKGNWKNVPSNYHYCPSSSDLNWHNDLIGTAIQVKMPASHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 15 | KFTIVFPHNQKGNWKNVPSNYHYCPSSSDLNWHNDLIGTAIQVKMPASHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 16 | KFTIVFPHNQKGNWKNVPSNYHYCPSSSDLNWHNDLIGTAIQVKMPASHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 17 | KFTIVFPHNQKGNWKNVPSNYHYCPSSSDLNWHNDLIGTALQVKMPASHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 18 | KFTIVFPHNQKGNWKNVPSNYHYCPSSSDLNWHNDLIGTALQVKMPASHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 19 | KFTIVFPHNQKGNWKNVPSNYHYCPSSSDLNWHNDLIGTALQVKMPASHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 20 | KFTIVFPHNQKGNWKNVPSNYHYCPSSSDLNWHNDLIGTALQVKMPASHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 21 | KFTIVFPHNQKGNWKNVPSNYHYCPSSSDLNWHNDLIGTAIQVKMPASHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 22 | KFTIVFPHNQKGNWKNVPSNYHYCPSSSDLNWHNDLIGTAIQVKMPASHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 23 | KFTIVFPHNQKGNWKNVPSNYHYCPSSSDLNWHNDLIGTAIQVKMPASHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 24 | KFTIVFPHNQKGNWKNVPSNYHYCPSSSDLNWHNDLIGTAIQVKMPASHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 25 | KFTIVFPHNQKGNWKNVPSNYHYCPSSSDLNWHNDLIGTALQVKMPASHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 26 | KFTIVFPHNQKGNWKNVPSNYHYCPSSSDLNWHNDLIGTALQVKMPASHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 27 | KFTIVFPHNQKGNWKNVPSNYHYCPSSSDLNWHNDLIGTALQVKMPASHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 28 | KFTIVFPHNQKGNWKNVPSNYHYCPSSSDLNWHNDLIGTALQVKMPASHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 29 | KFTIVFPHNQKGNWKNVPSNYHYCPSSSDLNWHNDLIGTAIQVKMPGSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 30 | KFTIVFPHNQKGNWKNVPSNYHYCPSSSDLNWHNDLIGTAIQVKMPGSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 31 | KFTIVFPHNQKGNWKNVPSNYHYCPSSSDLNWHNDLIGTAIQVKMPGSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 32 | KFTIVFPHNQKGNWKNVPSNYHYCPSSSDLNWHNDLIGTAIQVKMPGSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 33 | KFTIVFPHNQKGNWKNVPSNYHYCPSSSDLNWHNDLIGTALQVKMPGSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 34 | KFTIVFPHNQKGNWKNVPSNYHYCPSSSDLNWHNDLIGTALQVKMPGSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 35 | KFTIVFPHNQKGNWKNVPSNYHYCPSSSDLNWHNDLIGTALQVKMPGSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 36 | KFTIVFPHNQKGNWKNVPSNYHYCPSSSDLNWHNDLIGTALQVKMPGSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 37 | KFTIVFPHNQKGNWKNVPSNYHYCPSSSDLNWHNDLIGTAIQVKMPGSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 38 | KFTIVFPHNQKGNWKNVPSNYHYCPSSSDLNWHNDLIGTAIQVKMPGSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 39 | KFTIVFPHNQKGNWKNVPSNYHYCPSSSDLNWHNDLIGTAIQVKMPGSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 40 | KFTIVFPHNQKGNWKNVPSNYHYCPSSSDLNWHNDLIGTAIQVKMPGSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 41 | KFTIVFPHNQKGNWKNVPSNYHYCPSSSDLNWHNDLIGTALQVKMPGSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 42 | KFTIVFPHNQKGNWKNVPSNYHYCPSSSDLNWHNDLIGTALQVKMPGSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 43 | KFTIVFPHNQKGNWKNVPSNYHYCPSSSDLNWHNDLIGTALQVKMPGSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 44 | KFTIVFPHNQKGNWKNVPSNYHYCPSSSDLNWHNDLIGTAIQVKMPGSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 45 | KFTIVFPHNQKGNWKNVPSNYHYCPSSSDLNWHNDLIGTAIQVKMPFSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 46 | KFTIVFPHNQKGNWKNVPSNYHYCPSSSDLNWHNDLIGTAIQVKMPFSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 47 | KFTIVFPHNQKGNWKNVPSNYHYCPSSSDLNWHNDLIGTAIQVKMPFSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 48 | KFTIVFPHNQKGNWKNVPSNYHYCPSSSDLNWHNDLIGTAIQVKMPFSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLP ISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 49 | KFTIVFPHNQKGNWKNVPSNYHYCPSSSDLNWHNDLIGTALQVKMPFSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 50 | KFTIVFPHNQKGNWKNVPSNYHYCPSSSDLNWHNDLIGTALQVKMPFSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 51 | KFTIVFPHNQKGNWKNVPSNYHYCPSSSDLNWHNDLIGTALQVKMPFSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 52 | KFTIVFPHNQKGNWKNVPSNYHYCPSSSDLNWHNDLIGTALQVKMPFSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 53 | KFTIVFPHNQKGNWKNVPSNYHYCPSSSDLNWHNDLIGTAIQVKMPFSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 54 | KFTIVFPHNQKGNWKNVPSNYHYCPSSSDLNWHNDLIGTAIQVKMPFSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 55 | KFTIVFPHNQKGNWKNVPSNYHYCPSSSDLNWHNDLIGTAIQVKMPFSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 56 | KFTIVFPHNQKGNWKNVPSNYHYCPSSSDLNWHNDLIGTAIQVKMPFSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 57 | KFTIVFPHNQKGNWKNVPSNYHYCPSSSDLNWHNDLIGTALQVKMPFSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 58 | KFTIVFPHNQKGNWKNVPSNYHYCPSSSDLNWHNDLIGTALQVKMPFSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 59 | KFTIVFPHNQKGNWKNVPSNYHYCPSSSDLNWHNDLIGTALQVKMPFSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 60 | KFTIVFPHNQKGNWKNVPSNYHYCPSSSDLNWHNDLIGTALQVKMPFSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 61 | KFTIVFPHNQKGNWKNVPSNYHYCPSSSDLNWHNDLIGTAIQVKMPQSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 62 | KFTIVFPHNQKGNWKNVPSNYHYCPSSSDLNWHNDLIGTAIQVKMPQSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 63 | KFTIVFPHNQKGNWKNVPSNYHYCPSSSDLNWHNDLIGTAIQVKMPQSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 64 | KFTIVFPHNQKGNWKNVPSNYHYCPSSSDLNWHNDLIGTAIQVKMPQSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 65 | KFTIVFPHNQKGNWKNVPSNYHYCPSSSDLNWHNDLIGTALQVKMPQSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 66 | KFTIVFPHNQKGNWKNVPSNYHYCPSSSDLNWHNDLIGTALQVKMPQSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 67 | KFTIVFPHNQKGNWKNVPSNYHYCPSSSDLNWHNDLIGTALQVKMPQSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 68 | KFTIVFPHNQKGNWKNVPSNYHYCPSSSDLNWHNDLIGTALQVKMPQSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 69 | KFTIVFPHNQKGNWKNVPSNYHYCPSSSDLNWHNDLIGTAIQVKMPQSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 70 | KFTIVFPHNQKGNWKNVPSNYHYCPSSSDLNWHNDLIGTAIQVKMPQSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 71 | KFTIVFPHNQKGNWKNVPSNYHYCPSSSDLNWHNDLIGTAIQVKMPQSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 72 | KFTIVFPHNQKGNWKNVPSNYHYCPSSSDLNWHNDLIGTAIQVKMPQSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 73 | KFTIVFPHNQKGNWKNVPSNYHYCPSSSDLNWHNDLIGTALQVKMPQSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 74 | KFTIVFPHNQKGNWKNVPSNYHYCPSSSDLNWHNDLIGTALQVKMPQSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 75 | KFTIVFPHNQKGNWKNVPSNYHYCPSSSDLNWHNDLIGTALQVKMPQSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 76 | KFTIVFPHNQKGNWKNVPSNYHYCPSSSDLNWHNDLIGTALQVKMPQSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |

In particular embodiments, a vesiculovirus is cocal virus (COCV). In particular embodiments, a mutated viral envelope glycoprotein is derived from a COCV envelope glycoprotein (COCV-G; e.g., SEQ ID NO: 3: KFSIVFPQSQKGNWKNVPSSYHYCPSSSDQNWHNDLLGITMKVKMPKTHKAIQA DGWMCHAAKWITTCDFRWYGPKYITHSIHSIQPTSEQCKESIKQTKQGTWMSPGF PPQNCGYATVTDSVAVVVQATPHHVLVDEYTGEWIDSQFPNGKCETEECETVHN STVWYSDYKVTGLCDATLVDTEITFFSEDGKKESIGKPNTGYRSNYFAYEKGDKV CKMNYCKHAGVRLPSGVWFEFVDQDVYAAAKLPECPVGATISAPTQTSVDVSLI LDVERILDYSLCQETWSKIRSKQPVSPVDLSYLAPKNPGTGPAFTIINGTLKYFETR YIRIDIDNPIISKMVGKISGSQTERELWTEWFPYEGVEIGPNGILKTPTGYKFPLFMI GHGMLDSDLHKTSQAEVFEHPHLAEAPKQLPEEETLFFGDTGISKNPVELIEGWFS SWKSTVVTFFFAIGVFILLYVVARIVIA VRYRYQGSNNKRIYNDIEMSRFRK) or an amino acid sequence at least 95%, 96%, 97%, 98%, or 99% identical thereto comprising one or more modifications that enable the polypeptide to mediate fusion of the viral particle and a cell but that substantially ablate or ablate the polypeptide's ability to bind its cognate receptor expressed on a cell, e.g., LDL-R. In particular embodiments, a COCV-G envelope protein comprises one or more amino acid substitutions at K47 and/or R354. In particular embodiments, a COCV-G envelope protein comprises one or more amino acid substitutions at K47 and/or R354, wherein each amino acid can be substituted with A, G, F, or Q. In particular embodiments, a COCV-G envelope protein comprises one or more amino acid substitutions at K47 and/or R354 (with reference to a COCV-G envelope protein that lacks a signal peptide, e.g., SEQ ID NO: 3). In particular embodiments, a COCV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q. In particular embodiments, a COCV-G envelope protein comprises an amino acid sequence set forth in SEQ ID NO: 4 (wherein X₁=A and X₂=A; X₁=A and X₂=G; X₁=A and X₂=F; X₁=A and X₂=Q; X₁=G and X₂=A; X₁=G and X₂=G; X₁=G and X₂=F; X₁=G and X₂=Q; X₁=F and X₂=A; X₁=F and X₂=G; X₁=F and X₂=F; X₁=F and X₂=Q; X₁=Q and X₂=A; X₁=Q and X₂=G; X₁=Q and X₂=F; or X₁=A and X₂=Q) or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R.

TABLE 3

| SEQ ID | |
|---|---|
| NO | Amino Acid Sequence |
| 4 | KFSIVFPQSQKGNWKNVPSSYHYCPSSSDQNWHNDLLGITMK |
| | VKMPX₁THKAIQADGWMCHAAKWITTCDFRWYGPKYITHSIH |
| | SIQPTSEQCKESIKQTKQGTWMSPGFPPQNCGYATVTDSVAV |
| | VVQATPHHVLVDEYTGEWIDSQFPNGKCETEECETVHNSTVW |
| | YSDYKVTGLCDATLVDTEITFFSEDGKKESIGKPNTGYRSNY |
| | FAYEKGDKVCKMNYCKHAGVRLPSGVWFEFVDQDVYAAAKLP |
| | ECPVGATISAPTQTSVDVSLILDVERILDYSLCQETWSKIRS |
| | KQPVSPVDLSYLAPKNPGTGPAFTIINGTLKYFETRYIRIDI |
| | DNPIISKMVGKISGSQTEX₂ELWTEWFPYEGVEIGPNGILKT |
| | PTGYKFPLFMIGHGMLDSDLHKTSQAEVFEHPHLAEAPKQLP |
| | EEETLFFGDTGISKNPVELIEGWFSSWKSTVVTFFFAIGVFI |
| | LLYVVARIVIAVRYRYQGSNNKRIYNDIEMSRFRK |

In particular embodiments, a mutated COCV-G envelope protein comprises an amino acid sequence set forth in any one of SEQ ID NOs: 77-92 or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R. In particular embodiments, a mutated COCV-G envelope protein comprises an amino acid sequence set forth in any one of SEQ ID NOs: 89-92 or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R. In particular embodiments, a mutated VSIV-G envelope protein comprises an amino acid sequence set forth in SEQ ID NOs: 89 or 92 or an amino acid sequence at least 95%, 96%, 97%, 98%, or 99% identical thereto that mediates fusion of the viral particle and a 5 cell but that substantially ablates or ablates the polypeptide's ability to bind its cognate receptor expressed on a cell, e.g., LDL-R.

TABLE 4

| SEQ ID | |
|---|---|
| NO: | AMINO ACID SEQUENCE |
| 77 | KFSIVFPQSQKGNWKNVPSSYHYCPSSSDQNWHNDLLGITMKVKMPATHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEAELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 78 | KFSIVFPQSQKGNWKNVPSSYHYCPSSSDQNWHNDLLGITMKVKMPATHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEGELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 79 | KFSIVFPQSQKGNWKNVPSSYHYCPSSSDQNWHNDLLGITMKVKMPATHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEFELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 80 | KFSIVFPQSQKGNWKNVPSSYHYCPSSSDQNWHNDLLGITMKVKMPATHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEQELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 81 | KFSIVFPQSQKGNWKNVPSSYHYCPSSSDQNWHNDLLGITMKVKMPGTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEAELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 82 | KFSIVFPQSQKGNWKNVPSSYHYCPSSSDQNWHNDLLGITMKVKMPGTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEGELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 83 | KFSIVFPQSQKGNWKNVPSSYHYCPSSSDQNWHNDLLGITMKVKMPGTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEFELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 84 | KFSIVFPQSQKGNWKNVPSSYHYCPSSSDQNWHNDLLGITMKVKMPGTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEQELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 85 | KFSIVFPQSQKGNWKNVPSSYHYCPSSSDQNWHNDLLGITMKVKMPFTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEAELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 86 | KFSIVFPQSQKGNWKNVPSSYHYCPSSSDQNWHNDLLGITMKVKMPFTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEGELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 87 | KFSIVFPQSQKGNWKNVPSSYHYCPSSSDQNWHNDLLGITMKVKMPFTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEFELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 88 | KFSIVFPQSQKGNWKNVPSSYHYCPSSSDQNWHNDLLGITMKVKMPFTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEQELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 89 | KFSIVFPQSQKGNWKNVPSSYHYCPSSSDQNWHNDLLGITMKVKMPQTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEAELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 90 | KFSIVFPQSQKGNWKNVPSSYHYCPSSSDQNWHNDLLGITMKVKMPQTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEGELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 91 | KFSIVFPQSQKGNWKNVPSSYHYCPSSSDQNWHNDLLGITMKVKMPQTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEFELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 92 | KFSIVFPQSQKGNWKNVPSSYHYCPSSSDQNWHNDLLGITMKVKMPQTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEQELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |

In particular embodiments, one or more mutated morbillivirus envelope glycoproteins are derived from measles virus F (MV-F) and measles virus H (MV-H). In particular embodiments, a recombinant particle comprises one or more measles virus viral envelope glycoproteins modified to lack cell binding activity and retain fusogenic activity. In some embodiments, a recombinant particle comprises a modified MV-F glycoprotein and an MV-H glycoprotein modified to lack cell binding activity and retain fusogenic activity.

In particular embodiments, one or more mutated morbillivirus envelope glycoproteins are derived from measles virus F (MV-F) polypeptide (e.g., SEQ ID NO: 5: QIHWGNLSKIG VVGIGSASYKVMTRSSHQSLVIKLMPNITLLNNCTR VEIAEYRRLLRTVLEPIRDA LNAMTQNIRPVQSVASSRRHKRFAGVVLAGAALGVATAAQITAGIALHQSMLNS QAIDNLRASLETTNQAIEAIRQAGQEMILAVQGVODYINNELIPSMNQLSCDLIGQ KLGLKLLRYYTEILSLFGPSLRDPISAEISIQALSYALGGDINKVLEKLGYSGGDLL GILESRGIKARITHVDTESYFIVLSIAYPTLSEIKGVIVHRLEGVSYNIGSQEWYTTV PKYVATQGYLISNFDESSCTFMPEGTVCSQNALYPMSPLLQECLRGSTKSCARTL VSGSFGNRFILSQGNLIANCASILCKCYTTGTIINQDPDKILTYIAADHCPVVEVNG VTIQVGSRRYPDAVYLHRIDLGPPISLERLDVGTNLGNAIAKLEDAKELLESSDQIL RSMKGLSSTSIVYILIA VCLGGLIGIPALICCCRGR) or an amino acid sequence at least 95%, 96%, 97%, 98%, or 99% identical thereto and a measles virus H (MV-H) polypeptide (e.g., SEQ ID NO: 6: MGSRIVINREHLMIDRPYVLLA VLFVMFLSLIGLLAIAGIRLHRAAIYTAEIHKSLST NLDVTNSIEHQVKDVLTPLFKIIGDEVGLRTPQRFTDLVKFISDKIKFLNPDREYDF RDLTWCINPPERIKLDYDQYCADVAAEELMNALVNSTLLETRTTNQFLAVSKGN CSGPTTIRGQFSNMSLSLLDLYLGRGYNVSSIVTMTSQGMYGGTYLVEKPNLSSK RSELSQLSMYRVFEVGVIRNPGLGAPVFHMTNYLEQPVSNDLSNCMVALGELKL AALCHGEDSITIPYQGSGKGVSFQLVKLGVWKSPTDMQSWVPLSTDDPVIDRLYL SSHRGVIADNQAKWAVPTTRTDDKLRMETCFQQACKGKIQALCENPEWAPLKD NRIPSYGVLSVDLSLTVELKIKIASGFGPLITHGSGMDLYKSNHNNVYWLTIPPMK NLALGVINTLEWIPRFKVSPYLFTVPIKEAGGDCHAPTYLPAEVDGDVKLSSNLVI LPGQDLQYVLATYDTSRVEHAVVYYVYSPSRSFSYFYPFRLPIKGVPIELQVECFT WDQKLWCRHFCVLADSESGGHITHSGMVGMGVSCTVTREDGTN) or an amino acid sequence at least 95%, 96%, 97%, 98%, or 99% identical thereto, wherein the MV-H protein lacks cell binding activity and retains fusogenic activity. In particular embodiments, the MV-H polypeptide comprises one or more amino acid substitutions at positions Y463, R515, S530, and F531 of a MV-H polypeptide (e.g., SEQ ID NO 6). In particular embodiments, the MV-H polypeptide comprises one or more of the amino acid substitutions Y463A, R515A, S530L, and F531S in an MV-H polypeptide (e.g., SEQ ID NO: 7: MGSRIVINREHLMIDRPYVLLA VLFVMFLSLIGLLAIAGIRLHRAAIYTAEIHKSLST NLDVTNSIEHQVKDVLTPLFKIIGDEVGLRTPQRFTDLVKFISDKIKFLNPDREYDF RDLTWCINPPERIKLDYDQYCADVAAEELMNALVNSTLLETRTTNQFLAVSKGN CSGPTTIRGQFSNMSLSLLDLYLGRGYNVSSIVTMTSQGMYGGTYLVEKPNLSSK RSELSQLSMYRVFEVGVIRNPGLGAPVFHMTNYLEQPVSNDLSNCMVALGELKL AALCHGEDSITIPYQGSGKGVSFQLVKLGVWKSPTDMQSWVPLSTDDPVIDRLYL SSHRGVIADNQAKWAVPTTRTDDKLRMETCFQQACKGKIQALCENPEWAPLKD NRIPSYGVLSVDLSLTVELKIKIASGFGPLITHGSGMDLYKSNHNNVYWLTIPPMK NLALGVINTLEWIPRFKVSPALFNVPIKEAGGDCHAPTYLPAEVDGDVKLSSNLVI LPGQDLQYVLATYDTSAVEHAVVYYVYSPSRLSSYFYPFRLPIKGVPIELQVECFT WDQKLWCRHFCVLADSESGGHITHSGMVGMGVSCTVTREDGTN) or an amino acid sequence at least 95%, 96%, 97%, 98%, or 99% identical thereto.

In particular embodiments, one or more mutated henipaviruses envelope glycoproteins are derived from nipah virus F (NiV-F) and nipah virus G (NiV-G). In particular embodiments, a recombinant particle comprises one or more nipah virus viral envelope glycoproteins modified to lack cell binding activity and retain fusogenic activity. In some embodiments, a recombinant particle comprises a modified NiV-F glycoprotein and a NiV-G glycoprotein modified to lack cell binding activity and retain fusogenic activity.

In particular embodiments, one or more mutated henipavirus envelope glycoproteins are derived from a nipah virus F (NiV-F) polypeptide (e.g., SEQ ID NO: 8: LHYEKLSKIGLVKGVTRKYKIKSNPLTKDIVIKMIPNVSNMSQCTGSVMENYKTR LNGILTPIKGALEIYKNNTHDLVGDVRLAGVIMAGVAIGIATAAQITAGVALYEA MKNADNINKLKSSIESTNEAVVKLQETAEKTVYVLTALQDYINTNLVPTIDKISCK QTELSLDLALSKYLSDLLFVFGPNLQDPVSNSMTIQAISQAFGGNYETLLRTLGYA TEDFDDLLESDSITGQIIYVDLSSYYIIVRVYFPILTEIQQAYIQELLPVSENNDNSEW ISIVPNFILVRNTLISNIEIGFCLITKRSVICNQDYATPMTNNMRECLTGSTEKCPREL VVSSHVPRFALSNGVLFANCISVTCQCQTTGRAISQSGEQTLLMIDNTTCPTAVLG NVIISLGKYLGSVNYNSEGIAIGPPVFTDKVDISSQISSMNQSLQQSKDYIKEAQRL LDTVNPSLISMLSMIILYVLSIASLCIGLITFISFIIVEKKRNT) or an amino acid sequence at least 95%, 96%, 97%, 98%, or 99% identical thereto and a nipah virus G (NiV-G) polypeptide (e.g., SEQ ID NO: 9: MKKINEGLLDSKILSAFNTVIALLGSIVIIVMNIMIIQNYTRSTDNQAVIKDALQGIQ QQIKGLADKIGTEIGPKVSLIDTSSTITIPANIGLLGSKISQSTASINENVNEKCKFTL PPLKIHECNISCPNPLPFREYRPQTEGVSNLVGLPNNICLQKTSNQILKPKLISYTLP VVGQSGTCITDPLLAMDEGYFAYSHLERIGSCSRGVSKQRIIGVGEVLDRGDEVPS LFMTNVWTPPNPNTVYHCSAVYNNEFYYVLCAVSTVGDPILNSTYWSGSLMMT RLAVKPKSNGGGYNQHQLALRSIEKGRYDKVMPYGPSGIKQGDTLYFPAVGFLV RTEFKYNDSNCPITKCQYSKPENCRLSMGIRPNSHYILRSGLLKYNLSDGENPKVV FIEISDQRLSIGSPSKIYDSLGQPVFYQASFSWDTMIKFGDVLTVNPLVVNWRNNT VISRPGQSQCPRFNTCPEICWEGVYNDAFLIDRINWISAGVFLDSNQTAENPVFTV FKDNEILYRAQLASEDTNAQKTITNCFLLKNKIWCISLVEIYDTGDNVIRPKLFAV KIPEQCT) or an amino acid sequence at least 95%, 96%, 97%, 98%, or 99% identical thereto, wherein the NiV-G polypeptide lacks cell binding activity and retains fusogenic activity. In particular embodiments, the NiV-G polypeptide comprises one or more amino acid substitutions at positions E468, W471, Q497, and E500 of an NiV-G polypeptide (e.g., SEQ ID NO: 9). In particular embodiments, the NiV-G polypeptide comprises one or more of the amino acid substitutions E468A, W471A, Q497A, and E500A in an NiV-G polypeptide (e.g., SEQ ID NO: 10: MKKINEGLLDSKILSAFNTVIALLGSIVIIVMNIMIIQNYTRSTDNQAVIKDALQGIQ QQIKGLADKIGTEIGPKVSLIDTSSTITIPANIGLLGSKISQSTASINENVNEKCKFTL PPLKIHECNISCPNPLPFREYRPQTEGVSNLVGLPNNICLQKTSNQILKPKLISYTLP VVGQSGTCITDPLLAMDEGYFAYSHLERIGSCSRGVSKQRIIGVGEVLDRGDEVPS LFMTNVWTPPNPNTVYHCSAVYNNEFYYVLCAVSTVGDPILNSTYWSGSLMMT RLAVKPKSNGGGYNQHQLALRSIEKGRYDKVMPYGPSGIKQGDTLYFPAVGFLV RTEFKYNDSNCPITKCQYSKPENCRLSMGIRPNSHYILRSGLLKYNLSDGENPKVV FIEISDQRLSIGSPSKIYDSLGQPVFYQASFSWDTMIKFGDVLTVNPLVVNWRNNT VISRPGQSQCPRFNTCPAICAEGVYNDAFLIDRINWISAGVFLDSNATAANPVFTV FKDNEILYRAQLASEDTNAQKTITNCFLLKNKIWCISLVEIYDTGDNVIRPKLFAV KIPEQCT) or an amino acid sequence at least 95%, 96%, 97%, 98%, or 99% identical thereto, wherein the NiV-G polypeptide lacks cell binding activity and retains fusogenic activity.

In particular embodiments, a recombinant retrovirus contemplated herein comprises an outer surface, a lipid bilayer, cell membrane, or viral envelope comprising or expressing one or more non-viral membrane bound tropism polypeptides. A “tropism polypeptide” is a polypeptide that binds one or more antigens on a target host cell. A “non-viral membrane bound tropism polypeptide” is a polypeptide that binds one or more antigens on a target host cell; that is not native to, or derived from, either in whole or in part, a virus; and that is attached to a lipid bilayer, cell membrane, or viral envelope.

In particular embodiments, a non-viral membrane bound tropism polypeptide comprises an extracellular antigen targeting domain, a spacer polypeptide, and a transmembrane domain. In particular embodiments, an extracellular antigen targeting domain binds an antigen expressed on an immune effector cell. In particular embodiments, an extracellular antigen targeting domain comprises an antibody or antigen binding fragment thereof that binds an antigen expressed on the surface of an immune effector cell. In particular embodiments, an extracellular antigen targeting domain binds an antigen expressed on an immune effector cell.

In particular embodiments, the extracellular antigen targeting domain binds CD3δ, CD3ε CD3γ, CD4, CD5, CD7, CD8α, or CD8β. In particular embodiments, the extracellular antigen targeting domain comprises an scFv, a murine scFv, a humanized scFv, or a human scFv that binds CD3δ, CD3ε CD3γ, CD4, CD5, CD7, CD8α, or CD8β.

In particular embodiments, a tropism polypeptide comprises an anti-CD3ε antibody or antigen binding fragment thereof. Illustrative examples of anti-CD3ε antibodies or antigen binding fragments thereof suitable for using in particular embodiments include scFvs or other antigen binding fragments isolated from OKT3, UCHT1, YTH12.5, and TR66, and variants thereof, e.g., teplizumab and variants having at least 75%, at least 80%, at least 85%, at least 90%, at least 95%, or at least 99% identity thereto.

Illustrative anti-CD3 scFvs include the following amino acid sequences.

| SEQ ID | |
|---|---|
| NO: | Amino Acid Sequence |
| 11 | DIQMTQSPSSLSASVGDRVTITCSASSSVSYMNWYQQTPGKAP |
| | KRWIYDTSKLASGVPSRFSGSGSGTDYTFTISSLQPEDIATYY |
| | CQQWSSNPFTFGQGTKLQITRTSGGGGSGGGGSGGGGSQVQLV |
| | QSGGGVVQPGRSLRLSCKASGYTFTRYTMHWVRQAPGKGLEWI |
| | GYINPSRGYTNYNQKVKDRFTISRDNSKNTAFLOMDSLRPEDT |
| | GVYFCARYYDDHYCLDYWGQGTPVTVSS |
| 12 | DIQMTQSPSSLSASVGDRVTITCSASSSVSYMNWYQQTPGKAP |
| | KRWIYDTSKLASGVPSRFSGSGSGTDYTFTISSLQPEDIATYY |
| | CQQWSSNPFTFGQGTKLQITRTSGGGGSGGGGSGGGGSQVQLV |
| | QSGGGVVQPGRSLRLSCKASGYTFTRYTMHWVRQAPGKGLEWI |
| | GYINPSRGYTNYNQKVKDRFTISRDNSKNTAFLQMDSLRPEDT |
| | GVYFCARYYDDHYCLDYWGQGTPVTVSS |

In particular embodiments, a tropism polypeptide comprises a Gaussia luciferase signal peptide, an anti-CD3 scFv, a hinge domain (e.g., isolated from an IgG1 Fc region, an IgG1hinge or a human CD8 stalk, the mouse CD8a stalk, or Glycophorin A) or linker (e.g., a (G4S)₁₋₅), a transmembrane domain (e.g., isolated from CD3, CD4, CD8α, CD28, or Glycophorin A), and a cytoplasmic tail (e.g., isolated from Glycophorin A). Illustrative examples of polypeptides suitable for use in particular embodiments are disclosed in WO202107678 and WO2022164935, each of which is incorporated herein by reference in its entirety.

### D. Transduction Enhancers

In some embodiments, a recombinant retrovirus contemplated herein comprises a viral envelope comprising or expressing one or more transduction enhancers. A “transduction enhancer” as used herein refers to a non-viral membrane-bound protein that activates T cells. In particular embodiments, a transduction enhancer comprises a mitogenic and/or cytokine-based domain. In particular embodiments, a transduction enhancer comprises a mitogenic domain, a spacer domain, a transmembrane domain and a cytoplasmic tail. Illustrative examples of transduction enhancers are disclosed in WO202107678 and WO2022164935, each of which is incorporated herein by reference in its entirety.

A mitogenic domain is the part of the mitogenic transduction enhancer which causes T cell activation. It may bind or otherwise interact, directly or indirectly, with a T cell, leading to T cell activation. In particular embodiments, the mitogenic domain binds a T cell surface antigen including but not limited to the alpha or beta chains of a TCR, CD28, CD134 (OX40), CD137 (4-1BB), and CD278 (ICOS).

In particular embodiments, the mitogenic domain comprises an antibody or antigen binding fragment thereof that specifically binds a T cell surface antigen. In particular embodiments, the mitogenic domain comprises an antibody or antigen binding fragment thereof that specifically binds the alpha or beta chains of a TCR, CD28, CD134 (OX40), CD137 (4-1BB), and CD278 (ICOS).

Illustrative examples of anti-CD28 antibodies or antigen binding fragments thereof suitable for use in particular embodiments include those derived from 9.3, 15E8, CD28.2, 10F3 and TGN1412 and humanized variants thereof.

In particular embodiments, a mitogenic domain comprises all or part of a co-stimulatory molecule including but not limited to CD80, CD86, OX40L, 4-1BBL, and ICOSL.

In particular embodiments, a mitogenic transduction enhancer and/or cytokine-based transduction enhancer comprises a spacer domain that connects the mitogenic domain with the transmembrane domain. A flexible spacer allows the antigen binding domain to orient in different directions to facilitate binding. In particular embodiments, the spacer domain comprises an IgG1 Fc region, an IgG1 hinge, a human CD8a stalk or the mouse CD8a stalk, a CD2 ectodomain, or a CD34 ectodomain. In particular embodiments, the spacer domain comprises an alternative linker sequence which has similar length and/or domain spacing properties as an IgG1 Fc region, an IgG1 hinge or a CD8a stalk. In particular embodiments, a human IgG1 spacer may be altered to remove Fc binding motifs. In some embodiments, the spacer domain may be derived from a human protein.

In particular embodiments, the transduction enhancer comprises a transmembrane domain that tethers the transduction enhancer and/or cytokine-based transduction enhancer to the membrane. In particular embodiments, the transmembrane domain comprises a hydrophobic alpha helix. In particular embodiments, the transmembrane domain may be derived from CD3, CD4, CD8a, or CD28. In some embodiments, the transmembrane domain is derived from a human protein.

In some embodiments, a transduction enhancer does not comprise a transmembrane domain but instead comprises a membrane-targeting domain such as a GPI anchor. GPI anchoring is a post-translational modification which occurs in the endoplasmic reticulum. Preassembled GPI anchor precursors are transferred to proteins bearing a C-terminal GPI signal sequence. During processing, the GPI anchor replaces the GPI signal sequence and is linked to the target protein via an amide bond. The GPI anchor targets the mature protein to the membrane. In some embodiments, the present tagging protein comprises a GPI signal sequence.

### E. Engineered Antigen Receptors

In particular embodiments, a recombinant retrovirus contemplated herein are used to transduce a polynucleotide encoding one or more engineered antigen receptors into an immune effector cell. Engineered antigen receptors includes CARs and DARICs.

In particular embodiments, expression of the engineered antigen receptor is modulated by a degron fusion polypeptide wherein suppression of the degron fusion polypeptide is chemically inducible by a ligand. In certain embodiments, expression of the engineered antigen receptor is modulated by an FRB-degron fusion polypeptide and wherein suppression of the FRB-degron fusion polypeptide is chemically inducible by a ligand. In some embodiments, the ligand is rapamycin or a rapalog as described herein.

### 1. Chimeric Antigen Receptors

In particular embodiments, the engineered antigen receptor is a CAR. A “chimeric antigen receptor” or “CAR” refers to a synthetically designed receptor comprising an antigen binding domain of an antibody or other protein sequence that binds to a molecule expressed or displayed on a target cell (e.g., a cancer cell), a transmembrane domain, one or more intracellular signaling domains, e.g., one or more co-stimulatory domains and/or a primary signaling domain. The antigen binding domain is linked via a hinge domain to a transmembrane domain which is in turn linked, optionally by a spacer or linker domain, to one or more T cell intracellular signaling domains. CARs are engineered antigen receptors that can enable an immune receptor cell specifically recognize a target cell. In particular embodiments, the hinge or spacer/linker domain(s) are selected (e.g., for a particular length of amino acids) to achieve desired binding and/or functional characteristics of the CAR.

a. Antigen Binding Domain

In particular embodiments, the antigen binding fragment comprises a Camel Ig, a Llama Ig, an Alpaca Ig, Ig NAR, a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), a single-domain antibody (sdAb, a camelid VHH, Nanobody) or centyrin.

In particular embodiments, the antigen binding domain comprises an scFv or one or more VHHs.

In particular embodiments, the antigen binding domain binds CD19, CD20, CD22, CD3δ, CD79a, CD79b, GPCR5D, or BCMA.

In particular embodiments, the antigen binding domain binds CD19.

In particular embodiments, the antigen binding domain binds BCMA.

In particular embodiments, the antigen is expressed on a malignant B cell. In particular embodiments, the B cell malignancy is diffuse large B cell lymphoma (DLBCL) cell, Burkitt's type large B cell lymphoma (B-LBL) cell, follicular lymphoma (FL) cell, chronic lymphocytic leukemia (CLL) cell, acute lymphocytic leukemia (ALL) cell, mantle cell lymphoma (MCL), or multiple myeloma.

Illustrative anti-CD19 scFvs include the following amino acid sequence.

| SEQ ID | |
|---|---|
| NO: | Amino Acid Sequence |
| 93 | DIQMTQTTSSLSASLGDRVTISCRASQDISKYLNWYQQKPDGT |
| | VKLLIYHTSRLHSGVPSRFSGSGSGTDYSLTISNLEQEDIATY |
| | FCQQGNTLPYTFGGGTKLEITGSTSGSGKPGSGEGSTKGEVKL |
| | QESGPGLVAPSQSLSVTCTVSGVSLPDYGVSWIRQPPRKGLEW |
| | LGVIWGSETTYYNSALKSRLTIIKDNSKSQVFLKMNSLQTDDT |
| | AIYYCAKHYYYGGSYAMDYWGQGTSVTVSS |

b. Linkers

In particular embodiments, a CAR comprises a linker positioned between the extracellular domain and the transmembrane domain, optionally wherein the linker is about 2 to 100 amino acids in length. The linker can include or be composed of flexible residues such as glycine and serine so that the adjacent protein domains are free to move relative to one another. Longer linkers may be used, e.g., when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof.

In particular embodiments, the linker is P2A self-cleaving peptide, a T2A self-cleaving peptide, an E2A self-cleaving peptide, or an IgG4 hinge region polypeptide.

c. Transmembrane Domain

The transmembrane region can be any transmembrane region that can be incorporated into a functional CAR. Illustrative examples of transmembrane (TM) domains for use in CARs contemplated herein may be isolated, obtained, or derived from polypeptides including, but not limited to the alpha, beta, gamma, or delta chain of the T cell receptor, CD2, CD3, CD4, CD8α, and CD28

d. Intracellular Signaling Domain

In particular embodiments, a CAR comprises one or more intracellular signaling domains. In particular embodiments, a CAR comprises a costimulatory signaling domain and a primary signaling domain.

Illustrative examples of primary signaling domains suitable for use in CARs contemplated herein may be isolated, obtained, or derived from polypeptides including, but not limited to FcRγ, FcRβ, CD3ζ, CD79a, CD79b, and CD66d.

Illustrative examples of costimulatory domains suitable for use in CARs contemplated herein may be isolated, obtained, or derived from polypeptides including, but not limited to CD2, CD7, CD11a, CD27, CD28, CD30, CD40, CD134 (OX40), CD137 (4-1BB), CD278 (ICOS), and TNF receptor superfamily member 14 (TNFRS14).

In particular embodiments, the viral vector encodes a CAR comprising an antigen binding domain that binds CD19 or BCMA, an IgG4 linker, a CD28 derived transmembrane domain, a 4-1BB costimulatory domain, and a CD3ζ primary signaling domain.

2. DARICs

In particular embodiments, the engineered antigen receptor that binds CD19 or BCMA is a dimerization activated receptor initiation complex (DARIC). A DARIC provides a binding component and a signaling component that are each expressed as separate fusion proteins comprising an extracellular multimerization domain. The multimerization domains of the components associate by binding to a bridging factor (see U.S. Pat. Appl. No. 2016/0311901, which is incorporated herein by reference in its entirety). Although the bridging factor leads to formation of the DARIC system components, formation of the DARIC complex does not produce significant signaling on its own. The described DARIC complexes contemplated herein only initiate physiologically relevant signals when the DARIC binding component binds to a target antigen (e.g., CD19, CD20, CD22, CD3δ, CD79a, CD79b, GPCR5D, or BCMA) expressed or displayed on a target cell. In particular embodiments, a DARIC binding component comprises an antigen binding domain that binds to CD19 or BCMA.

### F. Modular Cell Surface Receptors

In particular embodiments, a recombinant retrovirus or lentivirus comprises a viral envelope comprising or expressing one or more mutated viral envelope glycoproteins that retain fusogenic activity and lack cognate receptor binding activity, a non-viral membrane-bound tropism polypeptide, and a recombinant retroviral vector comprising a polynucleotide encoding a promoter and a polynucleotide encoding one or more modular cell surface receptors. As used herein, “modular cell surface receptor” refers to one or more proteins that can be induced to activate T cells. In particular embodiments, a recombinant retrovirus or lentivirus transduces a host T cell with a recombinant retroviral vector comprising a polynucleotide encoding a modular cell surface receptor such that the T cell expresses said protein. The modular cell surface receptor may then be induced to activate the transduced T cell. In particular embodiments, the modular cell surface receptor is a drug-inducible modular cell surface receptor. In particular embodiments, the modular cell surface receptor forms a chemical-induced signaling complex. In particular embodiments, one or more modular cell surface receptor form an engineered complex that initiates a signal into the interior of a cell as a direct outcome of ligand-induced dimerization. In particular embodiments, a modular cell surface receptor is comprised within a homodimer (dimerization of two identical components) or a heterodimer (dimerization of two distinct components). In particular embodiments, a modular cell surface receptor protein complex comprises one or more a synthetic and/or natural components. Additional modular cell surface receptors that are suitable for particular embodiments contemplated herein include those disclosed in WO 2016/139463 and WO 2018/111834, each of which is incorporated herein by reference, in its entirety.

In particular embodiments, a modular cell surface receptor comprises a first and a second polynucleotide. In particular embodiments, a first polynucleotide encodes a first modular cell surface receptor complex component that comprises a first extracellular binding domain or portion thereof, a hinge domain, a transmembrane domain, and a signaling domain or portion thereof. In particular embodiments, a second polynucleotide encodes a second modular cell surface receptor complex component that comprises a second extracellular binding domain or a portion thereof, a hinge domain, a transmembrane domain, and a signaling domain or portions thereof. In some embodiments, the first and second components may be positioned such that when expressed, they dimerize in the presence of a bridging factor.

As used herein, “rapamycin activated cytokine receptor” or “RACR” refers to a modular cell surface receptor that inducibly generates an intracellular signal that promotes proliferation and/or activity of a cell in the presence of rapamycin. In particular embodiments, a RACR transduces an IL2-like signal in an immune effector cell in the presence of rapamycin through IL-2R intracellular domain(s) or variants thereof. In particular embodiments, a RACR comprises one or more modular cell surface receptor, each comprising a ligand inducible multimerization domain, a transmembrane domain and an endodomain. Illustrative examples of RACR polypeptides are disclosed in U.S. Patent Application Publication No. 2020/0392204, and PCT Pub. No. WO2021/076788, each of which is hereby incorporated by reference in its entirety.

In particular embodiments, the RACR comprises a first modular cell surface receptor comprising a first multimerization domain and a second modular cell surface receptor comprising a second multimerization domain, wherein the first multimerization domain and the second multimerization domain specifically bind to one another in response to a molecule.

A “multimerization domain,” as used herein, refers to a polypeptide that preferentially interacts or associates with another different polypeptide directly or via a bridging molecule, wherein the interaction of different multimerization domains substantially contributes to or efficiently promotes multimerization (i.e., the formation of a dimer, trimer, or multipartite complex, which may be a homodimer, heterodimer, homotrimer, heterotrimer, homomultimer, heteromultimer). A multimerization domain may be derived either from a natural, synthetic, semi-synthetic, or recombinant source. Illustrative examples of multimerization domains suitable for use in particular embodiments contemplated herein include an FKBP polypeptide (e.g., FKBP F36V, FKBP12), an FRB polypeptide (e.g., FRB T2098L), a calcineurin polypeptide, a cyclophilin polypeptide, a bacterial DHFR polypeptide, a PYL1 polypeptide, an ABI1 polypeptide, a GIB1 polypeptide, a GAI polypeptide, or variants thereof.

In particular embodiments, a RACR comprises a transmembrane domain that anchors a modular cell surface receptor to the plasma membrane of a cell. The transmembrane domain may be derived either from a natural, synthetic, semi-synthetic, or recombinant source. Illustrative examples of transmembrane domains suitable for use in particular embodiments contemplated herein include but are not limited to those isolated or obtained from CD4 or CD8α.

Association of the first and second modular cell surface receptor proteins is mediated by a bridging factor. A “bridging factor” refers to a molecule that associates with and that is disposed between two or more multimerization domains. Bridging factors include peptides, antibodies, small molecules, inorganic or organic compounds, and nucleic acid molecules. In particular embodiments, multimerization domains substantially contribute to or efficiently promote formation of a polypeptide complex only in the presence of a bridging factor. In particular embodiments, multimerization domains do not contribute to or do not efficiently promote formation of a polypeptide complex in the absence of a bridging factor. Illustrative examples of bridging factors suitable for use in particular embodiments contemplated herein include, but are not limited to rapamycin (sirolimus) or a rapalog thereof, coumermycin or a derivative thereof, gibberellin or a derivative thereof, abscisic acid (ABA) or a derivative thereof, methotrexate or a derivative thereof, cyclosporin A or a derivative thereof, FKCsA or a derivative thereof, trimethoprim (Tmp)-synthetic ligand for FKBP (SLF) or a derivative thereof, HaXS, TMP-HTag, and ABT-737 or functional derivatives thereof or any combination thereof.

In particular embodiments, a bridging factor is rapamycin or a rapamycin analog (rapalog).

Rapamycin analogs (rapalogs) include but are not limited to those disclosed in U.S. Pat. No. 6,649,595, which rapalog structures are incorporated herein by reference in their entirety. In certain embodiments, a bridging factor is a rapalog with substantially reduced immunosuppressive effect as compared to rapamycin. In a particular embodiment, the rapalog is AP21967 derivatives (also known as C-16-(S)-7-methylindolerapamycin, IC50=10 nM, a chemically modified non-immunosuppressive rapamycin analogue).

Other illustrative examples of rapalogs include but are not limited to FK1012, FK506, deforolimus, everolimus, novolimus, pimecrolimus, ridaforolimus, tacrolimus, temsirolimus, umirolimus, and zotarolimus.

In one embodiment, a first multimerization domain comprises FRB T2098L, a second multimerization domain comprises FKBP12, and the bridging factor is rapalog AP21967.

In another embodiment, a first multimerization domain comprises FRB, a second multimerization domain comprises FKBP12, and the bridging factor is rapamycin, temsirolimus or everolimus.

In another embodiment, a first multimerization domain and a second multimerization domain both comprise FKBP, and the bridging factor is FK1012.

In another embodiment, a first multimerization domain and a second multimerization domain both comprise FKBP F36V, and the bridging factor is AP1903.

Other illustrative examples of multimerization domain pairs suitable for use in RACRs contemplated in particular embodiments include but are not limited to include from FKBP and FRB, FKBP and calcineurin, FKBP and cyclophilin, FKBP and bacterial DHFR, calcineurin and cyclophilin, PYL1 and ABI1, or GIB1 and GAI, or variants thereof.

In particular embodiments, a RACR capable of transducing an IL2/IL15 signal comprises a first modular cell surface receptor comprising an IL-2Rγ endodomain and a second modular cell surface receptor comprising an IL-2Rβ endodomain.

In particular embodiments, a RACR capable of transducing an IL7 signal comprises a first modular cell surface receptor comprising an IL-2Rγ endodomain and a second modular cell surface receptor comprising an IL-7Rα endodomain.

In particular embodiments, a RACR capable of transducing an IL12 signal comprises a first modular cell surface receptor comprising an IL-12Rβ1 endodomain and a second modular cell surface receptor comprising an IL-12Rβ2 endodomain.

In particular embodiments, a RACR capable of transducing an IL21 signal comprises a first modular cell surface receptor comprising an IL-2Rγ endodomain, and a second modular cell surface receptor comprising an IL-21R endodomain.

In particular embodiments, a RACR capable of transducing a Type 1 Interferon (IFN) signal comprises a first modular cell surface receptor comprising an IFNRα1 endodomain, and a second modular cell surface receptor comprising an IFNRα2 endodomain.

### G. Cytosolic FRB

In particular embodiments, a vector comprises a polynucleotide sequence that confers resistance to an immunosuppressive agent. In particular embodiments, the polynucleotide that confers resistance to an immunosuppressive agent binds rapamycin. In particular embodiments, a polynucleotide that confers resistance to an immunosuppressive agent encodes a cytosolic (“naked”) FRB domain. The naked FRB domain is an approximately 100 amino acid domain extracted from the mTOR protein kinase. It is expressed in the cytosol as a freely diffusible soluble protein. The purpose of the FRB domain is to reduce the inhibitory effects of rapamycin on mTOR in the transduced cells, which should allow for consistent activation of transduced T cells and give them a proliferative advantage over native T cells.

In particular embodiments, the retroviral particle comprises a polypeptide comprising a cytosolic FRB domain that shares at least 75%, at least 80%, at least 85%, at least 90%, at least 95%, at least 99%, or 100% identity to SEQ ID NO: 94: EMWHEGLEEASRLYFGERNVKGMFEVLEPLHAMMERGPQTLKETSFNQAYGRD LMEAQEWCRKYMKSGNVKDLLQAWDLYYHVFRRISK.

### H. TGF-β Modulatory Receptors

Tumor cells secrete transforming growth factor β (TGF-β) as a means to modulate the tumor microenvironment to promote cancer progression. Decreasing TGF-β signaling in T cells increases their ability to infiltrate, proliferate, and mediate antitumor responses.

In particular embodiments, a recombinant retrovirus comprises a polynucleotide encoding a receptor that dampens or modulates the effect of TGF-β on a T cell transduced with a retroviral vector contemplated herein.

In particular embodiments, a recombinant retrovirus comprises a polynucleotide encoding a dominant negative TGF-β receptor (TGF-βR-DN). The TGF-βR-DN comprises TGF-βRI and TGF-βRII extracellular domains and a truncated intracellular domain, such that the receptor binds TGF-β but is unable to initiate downstream intracellular signaling.

In particular embodiments, a recombinant retrovirus comprises a polynucleotide encoding a TGF-β signal convertor. Illustrative examples of TGF-β signal convertors may be found in U.S. Patent App. Pub. No. 2019/0350974, which is incorporated herein by reference in its entirety.

### I. Retroviral Vectors

Recombinant retroviruses contemplated herein comprise a retroviral vector. The retroviral vector is based on a retrovirus genome that has been engineered to remove viral accessory proteins but leave elements intact for packaging, reverse transcription and integration. Illustrative examples of retroviral vectors include but are not limited to those described in Naldini et al., (1996a, 1996b, and 1998); Zufferey et al., (1997); Dull et al., 1998, U.S. Pat. Nos. 6,013,516; and 5,994,136, each of which are incorporated herein by reference in their entireties.

In particular embodiments, a retroviral vector comprises a polynucleotide sequence encoding, from 5′ to 3′: an MNDU3 promoter, a CAR that binds CD19, CD20, CD22, CD3δ, CD79a, CD79b, GPCR5D, or BCMA, a cytosolic FRB domain, a RACR cell-surface receptor, and a woodchuck hepatitis virus post-transcriptional regulatory element (WPRE).

In particular embodiments, a retroviral vector comprises a polynucleotide sequence encoding, from 5′ to 3′: a CAR that binds CD19, CD20, CD22, CD3δ, CD79a, CD79b, GPCR5D, or BCMA, a cytosolic FRB domain, and a RACR cell-surface receptor.

In particular embodiments, a retroviral vector comprises a polynucleotide sequence encoding, from 5′ to 3′: an MNDU3 promoter, a cytosolic FRB domain, a RACR cell-surface receptor, a CAR that binds CD19, CD20, CD22, CD3δ, CD79a, CD79b, GPCR5D, or BCMA, and a WPRE.

In particular embodiments, a retroviral vector comprises a polynucleotide sequence encoding, from 5′ to 3′: a cytosolic FRB domain, a RACR cell-surface receptor, and a CAR that binds CD19, CD20, CD22, CD3δ, CD79a, CD79b, GPCR5D, or BCMA.

In particular embodiments, a retroviral vector comprises a polynucleotide sequence encoding, from 5′ to 3′: an MNDU3 promoter, a cytosolic FRB domain, a CAR that binds CD19, CD20, CD22, CD3δ, CD79a, CD79b, GPCR5D, or BCMA, a TGF-β-DN or TGF-β signal convertor; and a WPRE.

In particular embodiments, a retroviral vector comprises a polynucleotide sequence encoding, from 5′ to 3′: a cytosolic FRB domain, a CAR that binds CD19, CD20, CD22, CD3δ, CD79a, CD79b, GPCR5D, or BCMA, and a TGF-β-DN or TGF-β signal convertor.

In particular embodiments, a recombinant retrovirus comprises a polynucleotide sequence encoding a retroviral vector comprising, from 5′ to 3′: a 5′ LTR, an HIV-1 packaging signal (Psi), an HIV-1 Rev response element (RRE), a cPPT/CTS, an MNDU3 promoter, a human CSF2R signal peptide, an scFv that binds CD19, CD20, CD22, CD3δ, CD79a, CD79b, GPCR5D, or BCMA, an IgG4 hinge domain, a human CD28 transmembrane domain, a 4-1BB costimulatory domain, a CD3ζ primary signaling domain, a P2A sequence, a cytosolic FRB domain, a P2A sequence, an NGAL signal peptide, an FKBP12 polypeptide, an IL2Rγ transmembrane and cytoplasmic domain, a P2A sequence, a CD8α signal peptide, an FRB T2098L polypeptide, an IL2Rβ transmembrane and cytoplasmic domain, a WPRE, a 3′ SIN LTR, and optionally a poly(A) tail.

In particular embodiments, a recombinant retrovirus comprises a polynucleotide sequence encoding a retroviral vector comprising, from 5′ to 3′: a 5′ LTR, an HIV-1 packaging signal (Psi), an HIV-1 Rev response element (RRE), a cPPT/CTS, an MNDU3 promoter, a human CSF2R signal peptide, an scFv that binds CD19, CD20, CD22, CD3δ, CD79a, CD79b, GPCR5D, or BCMA, an IgG4 hinge domain, a human CD28 transmembrane domain, a 4-1BB costimulatory domain, a CD3ζ primary signaling domain, a P2A sequence, a cytosolic FRB domain, a P2A sequence, an NGAL signal peptide, an FKBP12 polypeptide, an IL2Rγ transmembrane and cytoplasmic domain, a P2A sequence, a CD8α signal peptide, an FRB T2098L polypeptide, an IL2Rβ transmembrane and cytoplasmic domain, a 3′ SIN LTR, and optionally a poly(A) tail.

In particular embodiments, a recombinant retrovirus comprises one or more components of a gene editing payload including but not limited to polynucleotides intended for insertion into the genome of the target cell and/or gene editing systems (CRISPR-Cas, meganucleases, homing endonucleases, zinc finger enzymes and the like).

### J. Immune Effector Cells

In particular embodiments, a recombinant retrovirus contemplated herein is engineered to transduced immune effector cells. Non-limiting examples of immune effector cells that can be transduced with the recombinant retrovirus contemplated herein include but are not limited to T lymphocytes, B lymphocytes, dendritic cells (DC), Treg cells, natural killer (NK) cells, natural killer T (NKT) cells, and macrophages.

T lymphocytes (“T cells”) are a type of lymphocyte (itself a type of white blood cell) that play a central role in cell-mediated immunity. Primary T cells include but are not limited to naïve T cells, MHC-restricted T cells, CD3⁺ T cells, CD4⁺ T cells, CD8⁺ T cells, or tumor infiltrating lymphocytes (TILs). T cells may be autologous or allogeneic.

Natural killer (NK) cells are cytotoxic lymphocytes that constitute a major component of the innate immune system. NK cells do not express T cell antigen receptors (TCR), CD3 or surface immunoglobulins (Ig) B cell receptor, but usually express the surface markers CD16 (FcyRIII) and CD56 in humans.

### K. Pharmaceutical Compositions and Formulations

Formulations and compositions contemplated herein comprise a combination of any number of recombinant retroviruses, polypeptides, polynucleotides, small molecules and formulated in pharmaceutically acceptable or physiologically-acceptable compositions for administration to a cell, tissue, organ, or an animal, either alone, or in combination with one or more other modalities of therapy. In particular embodiments, the one or more additional pharmaceutical agents further increases transduction efficiency of a recombinant retrovirus.

In particular embodiments a composition comprises a therapeutically effective amount of a retroviral particle formulated with one or more pharmaceutically acceptable carriers, diluents, excipients or stabilizers, in the form of lyophilized formulations or aqueous solutions. In some embodiments, one or more pharmaceutically acceptable surface-active agents (surfactant), buffers, isotonicity agents, salts, amino acids, sugars, stabilizers and/or antioxidant are used in the formulation.

In some embodiments, the composition further comprises other agents, such as, e.g., cytokines, growth factors, hormones, small molecules or various pharmaceutically active agents.

Suitable pharmaceutically acceptable surfactants comprise but are not limited to polyethylen-sorbitan-fatty acid esters, polyethylene-polypropylene glycols, polyoxyethylene-stearates and sodium dodecyl sulphates. Suitable buffers comprise but are not limited to histidine-buffers, citrate-buffers, succinate-buffers, acetate-buffers and phosphate-buffers.

Isotonicity agents are used to provide an isotonic formulation. Suitable isotonicity agents comprise but are not limited to salts, including but not limited to sodium chloride (NaCl) or potassium chloride, sugars including but not limited to glucose, sucrose, trehalose or and any component from the group of amino acids, sugars, salts and combinations thereof. In some embodiments, isotonicity agents are generally used in a total amount of about 5 mM to about 350 mM.

“Pharmaceutically acceptable” refers to molecular entities and compositions that do not produce an allergic or similar untoward reaction when administered to a human.

The term “carrier” includes any and all solvents, dispersion media, vehicles, coatings, diluents, antibacterial and antifungal agents, isotonic and absorption delaying agents, buffers, carrier solutions, suspensions, colloids, and the like.

A “pharmaceutically acceptable carrier” includes any and all solvents, dispersion media, coatings, antibacterial and antifungal agents, isotonic and absorption delaying agents, and the like that are physiologically compatible, including pharmaceutically acceptable cell culture media. In some embodiments, a composition comprising a carrier is suitable for parenteral administration, e.g., intravascular (intravenous or intraarterial), intraperitoneal or intramuscular administration. Pharmaceutically acceptable carriers include sterile aqueous solutions or dispersions and sterile powders for the extemporaneous preparation of sterile injectable solutions or dispersion.

The compositions may further comprise one or more polypeptides, polynucleotides, or compounds that increase the transduction efficiency, formulated in pharmaceutically acceptable or physiologically-acceptable solutions for administration to a cell or an animal, either alone, or in combination with one or more other modalities of therapy.

The pharmaceutical compositions that contain a recombinant retrovirus comprising an expression cassette or vector may be in any form that is suitable for the selected mode of administration, for example, for intraventricular, intramyocardial, intracoronary, intravenous, intra-arterial, intra-renal, intraurethral, epidural, intrathecal, intraperitoneal, or intramuscular administration. The retrovirus can be administered, as sole active agent, or in combination with other active agents, in a unit administration form, as a mixture with conventional pharmaceutical supports, to animals and human beings. In some embodiments, the pharmaceutical composition comprises cells transduced ex vivo with any of the particles contemplated herein.

In particular embodiments, a recombinant retrovirus (e.g., lentivirus), or a pharmaceutical composition comprising that recombinant retrovirus, is effective when administered systemically (e.g., intravenously). In certain embodiments, the retroviral vectors induce expression of CAR in transduced immune cells when a recombinant retrovirus is administered systemically.

In various embodiments, the pharmaceutical compositions contain vehicles (e.g., carriers, diluents and excipients) that are pharmaceutically acceptable for a formulation capable of being injected. Exemplary excipients include a poloxamer. Formulation buffers for viral particles generally contain salts to prevent aggregation and other excipients (e.g., poloxamer) to reduce stickiness of the viral particle. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. In particular embodiments, the formulation is stable for storage and use when frozen (e.g., at less than 0° C., about −60° C., or about −72° C.). In some embodiments, the formulation is a cryopreserved solution.

In particular embodiments, compositions are formulated for in vivo administration.

The pharmaceutical compositions of the present disclosure, formulation of pharmaceutically acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, intraperitoneal, and intramuscular administration and formulation. In certain embodiments, compositions contemplated herein are administered parenterally, intravenously, intramuscularly, or intraperitoneally.

A recombinant retrovirus may be used to transduce cells in vivo at an any effective dosage. In particular embodiments, a recombinant retrovirus is administered to a subject in vivo, by direct injection to the cell, tissue, organ or subject in need of therapy.

A recombinant retrovirus may also be delivered according to viral titer (TU/mL). The amount of lentivirus directly injected is determined by total TU and can vary based on both the volume that could be feasibly injected to the site and the type of tissue to be injected. In particular embodiments, the viral titer delivered is about 1×10⁹ to about 1×10¹² TU. Systemic delivery may accommodate a much larger TU, a load of about 1×10¹⁰ to about 1×10¹⁵.

In particular embodiments, a recombinant retrovirus is administered at a dose of between about 1×10¹² and 5×10¹⁵ vector genomes (vg) of the vector per kilogram (vg) of total body mass of the subject (vg/kg). In particular embodiments, a recombinant retrovirus is administered at a dose of between about 1×10¹² and 5×10¹⁵ vector particles (vp) of the vector per kilogram (vp) of total body mass of the subject (vp/kg).

Rapamune® (sirolimus, rapamycin) is available as an oral solution or tablet and is FDA approved. Per the US Prescribing Information (USPI), rapamycin is available in 1 mg/mL oral solution or 0.5, 1, or 2 mg tablets and is to be administered once daily. Rapamycin may also be delivered in other dosage forms and/or by other administration routes. In particular embodiments, rapamycin is administered at a dose of between about 0.1 mg/m² and 100 mg/m² of surface area of the subject. In particular embodiments, rapamycin is administered at a dose of between about 0.001 mg/m² and 100 mg/m² of surface area of the subject.

In particular embodiments, a dose of rapamycin is administered every day. In certain embodiments, a dose of rapamycin is administered about every 1, 2, 3, 4, 5, 6, 7, 8, 9, 10, 11, 12, 13, or 14 days. In some embodiments, a dose of rapamycin is administered about every 1, 2, 3, 4, 5, 6, 7, 8, 9, or 10 weeks. In other embodiments, a dose of rapamycin is administered about every 1, 2, 3, 4, 5, 6, 7, 8, 9, or 10 months.

In particular embodiments, a recombinant retrovirus is administered and a first dose of rapamycin is administered about 1, 2, 3, 4, 5, 6, 7, 8, 9, 10, 11, 12, 13, or 14 days, weeks, or months post administration of the retroviral particle.

In particular embodiments, administration of rapamycin increases the number immune effector cells transduced by a recombinant retrovirus. In certain embodiments, administration of rapamycin increases the number immune effector cells transduced by a recombinant retrovirus by at least 10%, at least 20%, at least 30%, at least 40%, at least 50%, at least 60%, at least 70%, at least 80%, at least 90%, at least 1-fold, at least 2-fold, at least 3-fold, at least 5-fold, at least 7-fold, or at least 10-fold, in the subject. In particular embodiments, the increase is evaluated by the number of transduced immune effector cells 1, 2, 3, 4, 5, 6, 7, 8, 9, 10, 11, or 12 weeks or months after the first dose of the rapamycin (once the virus is administered), wherein the reference number is the number of transduced immune cells on the day of the first dose of rapamycin.

In particular embodiments, a recombinant retrovirus is administered via a route selected from the group consisting of parenteral, intravenous, intramuscular, subcutaneous, intratumoral, intraperitoneal, and intralymphatic. In particular embodiments, a recombinant retrovirus is administered multiple times.

In particular embodiments, a recombinant retrovirus is administered by intraperitoneal, subcutaneous, or intranodal injection.

In particular embodiments, a recombinant retrovirus is administered as a single injection. In some embodiments, the retroviral particle is administered as at least 1, at least 2, at least 3, at least 4, at least 5, at least 6, at least 7, at least 8, at least 9, or at least 10 injections.

### L. Methods of Treatment

In various embodiments, a recombinant retrovirus and compositions contemplated herein are used to treat, prevent, or ameliorate at least one symptom of a disease, disorder, or condition. In particular embodiments, the disease, disorder, or condition is cancer. In particular embodiments, the cancer is a hematological malignancy or a solid tumor. In particular embodiments, the subject has relapsed or is refractory to treatment with a prior anti-cancer therapeutic.

In some embodiments, the cancer is a hematological malignancy.

In particular embodiments, a recombinant retrovirus contemplated herein treats, prevents, or ameliorates at least one symptom of a multiple myeloma, a smoldering multiple myeloma, a monoclonal gammopathy of undetermined significance (MGUS), an acute lymphoblastic leukemia (ALL), a diffuse large B cell lymphoma (DLBCL), a Burkitt's lymphoma (BL), a follicular lymphoma (FL), a mantle-cell lymphoma (MCL), Waldenstrom's macroglobulinema, a plasma cell leukemia, a light chain amyloidosis (AL), a precursor B cell lymphoblastic leukemia, a precursor B cell lymphoblastic leukemia, an acute myeloid leukemia (AML), a myelodysplastic syndrome (MDS), a chronic lymphocytic leukemia (CLL), a B cell malignancy, a chronic myeloid leukemia (CML), a hairy cell leukemia (HCL), a blastic plasmacytoid dendritic cell neoplasm, Hodgkin's lymphoma, nonHodgkin's lymphoma, a marginal zone B cell lymphoma (MZL), a mucosa-associated lymphatic tissue lymphoma (MALT), plasma cell leukemia, anaplastic large-cell lymphoma (ALCL), leukemia or lymphoma.

In particular embodiments, a recombinant retrovirus contemplated herein treats, prevents, or ameliorates at least one symptom of a CD19-expressing B cell malignancy.

In particular embodiments, a recombinant retrovirus contemplated herein treats, prevents, or ameliorates at least one symptom of a BCMA-expressing B cell malignancy.

In particular embodiments, a recombinant retrovirus contemplated herein treats, prevents, or ameliorates at least one symptom of a solid tumor.

In particular embodiments, a recombinant retrovirus contemplated herein treats, prevents, or ameliorates at least one symptom of prostate cancer, lung cancer, non-small cell lung cancer (NSCLC), liver cancer, cervical cancer, colon cancer, breast cancer, ovarian cancer, endometrial cancer, pancreatic cancer, melanoma, esophageal cancer, gastric cancer, stomach cancer, renal carcinoma, bladder cancer, hepatocellular carcinoma, renal cell carcinoma, urothelial carcinoma, head and neck cancer, glioma, glioblastoma, colorectal cancer, thyroid cancer, epithelial cancers, or adenocarcinomas.

In particular embodiments, a recombinant retrovirus is administered in combination with one or more anti-cancer therapies including, but not limited to, an autologous stem cell transplant (ASCT), radiation, surgery, a chemotherapeutic agent, an immunomodulatory agent and a targeted cancer therapy.

In particular embodiments, the one or more anti-cancer therapies is selected from the group consisting of 6-mercaptopurine, abiraterone, alemtuzumab, all-trans retinoic acid, anastrozole, aprepitant, arsenic trioxide, atezolizumab, azacytidine, bafetinib, bevacizumab, bleomycin, bortezomib, bosutinib, cabazitaxel, capecitabine, carboplatin, carfilzomib, cetuximab, cisplatin, cladribine, corticosteroid, crizotinib, cyclophosphamide, cytarabine, dasatinib, daunorubicin, danusertib, decitabine, denosumab, dexamethasone, docetaxel, doxorubicin, elotozumab, eribulin, erlotinib, etoposide, everolimus, exemestane, filgrastim, fludarabine, fluorouracil, fulvestrant, gemcitabine, hydroxyurea, idarubicin, imatinib, imiquimod, ipilimumab, ixabepilone, ixazomib, lapatinib, lenalidomide, letrozole, leuprolide, melphalan, methotrexate, mitoxantrone, nilotinib, nivolumab, oxaliplatin, paclitaxel, palonosetron, pembrolizumab, pemetrexed, pomalidomide, ponatinib, prednisone, radium-223, rituximab, saracatinib, sipuleucel-T, sorafenib, sunitinib, tamoxifen, temozolomide, temsirolimus, thalidomide, tinorelbine, topotecan, tozasertib, trastuzumab, vincristine, and zoledronic acid.

### M. Definitions

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description is for the purpose of describing particular embodiments only and is not intended to be limiting.

The singular articles “a”, “an”, and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (or).

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

The term “viral particle” as used herein refers a macromolecular complex capable of delivering a foreign nucleic acid molecule into a cell independent of another agent. A particle can be a viral particle or non-viral particle. Viral particle includes retroviral particle and lentiviral particle. Non-viral particles are limited to liposomes, nanoparticles, extracellular vesicles, microvesicles, virus-like particles, and the like.

The term “transduce” refers to introduction of a nucleic acid into a cell or host organism by way of a virus (e.g., a lentivirus). Introduction of a transgene into a cell a recombinant retrovirus can therefore be referred to as “transduction” of the cell. The transgene may or may not be integrated into the genomic nucleic acid of a transduced cell. If an introduced transgene becomes integrated into the nucleic acid (genomic DNA) of the recipient cell or organism it can be stably maintained in that cell. Alternatively, the introduced transgene may exist in the recipient cell or host organism extra-chromosomally, or only transiently. A “transduced cell” is therefore a cell into which the transgene has been introduced by way of transduction. Thus, a “transduced” cell is a cell into which, a polynucleotide has been introduced.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

The section headings used herein are for organizational purposes only and are not to be construed as limiting the subject matter described.

While illustrative embodiments have been illustrated and described, it will be appreciated that various changes can be made therein without departing from the spirit and scope of the invention.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A recombinant retrovirus comprising:
(a) a viral envelope comprising (i) one or more mutated viral envelope glycoproteins that retain fusogenic activity and lack cognate receptor binding activity and (ii) a non-viral membrane-bound tropism polypeptide, and
(b) a recombinant retroviral vector comprising a polynucleotide encoding a promoter operably linked to a polynucleotide encoding a chimeric antigen receptor (CAR) that binds CD19, CD20, CD22, CD3δ, CD79a, CD79b, GPCR5D, or BCMA and a polynucleotide encoding a modular cell surface receptor;
wherein the recombinant retrovirus transduces an immune effector cell in vivo.

2. The recombinant retrovirus of claim 1, wherein the recombinant retrovirus is a recombinant lentivirus.

3. The recombinant retrovirus of claim 1 or claim 2, wherein the recombinant retrovirus is a recombinant lentivirus selected from the group consisting of: human immunodeficiency virus 1 (HIV-1), human immunodeficiency virus 2 (HIV-2), visna-maedi virus (VMV), caprine arthritis-encephalitis virus (CAEV), equine infectious anemia virus (EIAV), feline immunodeficiency virus (FIV), bovine immune deficiency virus (BIV), and simian immunodeficiency virus (SIV).

4. The recombinant retrovirus of any one of claims 1 to 3, wherein the immune effector cell is selected from the group consisting of: a B lymphocyte, a T lymphocyte, a natural killer (NK) cell, and a natural killer T (NKT) cell.

5. The recombinant retrovirus of claim 4, wherein the T lymphocyte is an αβ T lymphocyte or a γδ T cell.

6. The recombinant retrovirus of any one of claims 1 to 5, wherein the one or more mutated viral envelope glycoproteins comprise a vesiculovirus envelope glycoprotein, one or more morbillivirus envelope glycoproteins or one or more henipavirus envelope glycoproteins.

7. The recombinant retrovirus of claim 6, wherein the vesiculovirus is selected from the group consisting of: vesicular stomatitis Alagoas virus (VSAV; Alagoas vesiculovirus), Carajás virus (CJSV; Carajas vesiculovirus), Chandipura virus (CHPV; Chandipura vesiculovirus), Cocal virus (COCV; Cocal vesiculovirus), vesicular stomatitis Indiana virus (VSIV; Indiana vesiculovirus), Isfahan virus (ISFV; Isfahan vesiculovirus), Maraba virus (MARAV; Maraba vesiculovirus), Morreton virus (MORV; Morreton vesiculovirus), vesicular stomatitis New Jersey virus (VSNJV; New Jersey vesiculovirus), and Piry virus (PIRYV; Piry vesiculovirus).

8. The recombinant retrovirus of claim 6 or claim 7, wherein the vesiculovirus envelope glycoprotein is a vesiculovirus G protein.

9. The recombinant retrovirus of any one of claims 6 to 8, wherein the vesiculovirus G protein is a COCV G glycoprotein (COCV-G) or a VSIV G glycoprotein (VSIV-G).

10. The recombinant retrovirus of claim 9, wherein the VSIV-G envelope protein comprises one or more of:
(a) one or more amino acid substitutions at H8, N9, Q10, K47, K50, A51, S183, S179, N180, 1182, M184, Y209, 1347, T350, T352, E353, and R354;
(b) an insertion of TT between N9 and Q10, an insertion of GGS between H8 and N9, an insertion of GGS between N9 and Q10, an insertion of TT between N208 and Y209, an insertion of GGS between P46 and K47, and an insertion of GGS between N208 and Y209; or
(c) amino acid substitutions at K47 and/or R354; or
(d) a deletion of residues 1-8.

11. The recombinant retrovirus of claim 9 or claim 10, wherein the VSIV-G envelope protein comprises one or more amino acid substitutions at H8, K47, Y209, and R354.

12. The recombinant retrovirus of any one of claims 9 to 11, wherein the VSIV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q.

13. The recombinant retrovirus of any one of claims 9 to 12, wherein the VSIV-G envelope protein comprises the amino acid sequence set forth in SEQ ID NO: 2, wherein X₁=I, X₂=A, X₃=Q, and X₄=A; X₁=I, X₂=A, X₃=Q, and X₄=G; X₁=I, X₂=A, X₃=Q, and X₄=F; X₁=I, X₂=A, X₃=Q, and X₄=Q; X₁=L, X₂=A, X₃=Q, and X₄=A; X₁=L, X₂=A, X₃=Q, and X₄=G; X₁=L, X₂=A, X₃=Q, and X₄=F; X₁=L, X₂=A, X₃=Q, and X₄=Q; X₁=I, X₂=A, X₃=H, and X₄=A; X₁=I, X₂=A, X₃=H, and X₄=G; X₁=I, X₂=A, X₃=H, and X₄=F; X₁=I, X₂=A, X₃=H, and X₄=Q; X₁=L, X₂=A, X₃=H, and X₄=A; X₁=L, X₂=A, X₃=H, and X₄=G; X₁=L, X₂=A, X₃=H, and X₄=F; X₁=L, X₂=A, X₃=H, and X₄=Q; X₁=I, X₂=G, X₃=Q, and X₄=A; X₁=I, X₂=G, X₃=Q, and X₄=G; X₁=I, X₂=G, X₃=Q, and X₄=F; X₁=I, X₂=G, X₃=Q, and X₄=Q; X₁=L, X₂=G, X₃=Q, and X₄=A; X₁=L, X₂=G, X₃=Q, and X₄=G; X₁=L, X₂=G, X₃=Q, and X₄=F; X₁=L, X₂=G, X₃=Q, and X₄=Q; X₁=I, X₂=G, X₃=H, and X₄=A; X₁=I, X₂=G, X₃=H, and X₄=G; X₁=I, X₂=G, X₃=H, and X₄=F; X₁=I, X₂=G, X₃=H, and X₄=Q; X₁=L, X₂=G, X₃=H, and X₄=A; X₁=L, X₂=G, X₃=H, and X₄=G; X₁=L, X₂=G, X₃=H, and X₄=F; X₁=L, X₂=G, X₃=H, and X₄=Q; X₁=I, X₂=F, X₃=Q, and X₄=A; X₁=I, X₂=F, X₃=Q, and X₄=G; X₁=I, X₂=F, X₃=Q, and X₄=F; X₁=I, X₂=F, X₃=Q, and X₄=Q; X₁=L, X₂=F, X₃=Q, and X₄=A; X₁=L, X₂=F, X₃=Q, and X₄=G; X₁=L, X₂=F, X₃=Q, and X₄=F; X₁=L, X₂=F, X₃=Q, and X₄=Q; X₁=I, X₂=F, X₃=H, and X₄=A; X₁=I, X₂=F, X₃=H, and X₄=G; X₁=I, X₂=F, X₃=H, and X₄=F; X₁=I, X₂=F, X₃=H, and X₄=Q; X₁=L, X₂=F, X₃=H, and X₄=A; X₁=L, X₂=F, X₃=H, and X₄=G; X₁=L, X₂=F, X₃=H, and X₄=F; X₁=L, X₂=F, X₃=H, and X₄=Q; X₁=I, X₂=Q, X₃=Q, and X₄=A; X₁=I, X₂=Q, X₃=Q, and X₄=G; X₁=I, X₂=Q, X₃=Q, and X₄=F; X₁=I, X₂=Q, X₃=Q, and X₄=Q; X₁=L, X₂=Q, X₃=Q, and X₄=A; X₁=L, X₂=Q, X₃=Q, and X₄=G; X₁=L, X₂=Q, X₃=Q, and X₄=F; X₁=L, X₂=Q, X₃=Q, and X₄=Q; X₁=I, X₂=Q, X₃=H, and X₄=A; X₁=I, X₂=Q, X₃=H, and X₄=G; X₁=I, X₂=Q, X₃=H, and X₄=F; X₁=I, X₂=Q, X₃=H, and X₄=Q; X₁=L, X₂=Q, X₃=H, and X₄=A; X₁=L, X₂=Q, X₃=H, and X₄=G; X₁=L, X₂=Q, X₃=H, and X₄=F; and X₁=L, X₂=Q, X₃=H, and X₄=Q.

14. The recombinant retrovirus of any one of claims 6 to 8, wherein the vesiculovirus G protein is COCV-G.

15. The recombinant retrovirus of claim 14, wherein the COCV-G envelope protein comprises one or more amino acid substitutions at K47 and R354.

16. The recombinant retrovirus of claim 14 or claim 15, wherein the COCV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q.

17. The recombinant retrovirus of any one of claims 14 to 16, wherein the COCV-G envelope protein comprises the amino acid sequence set forth in SEQ ID NO: 4, wherein X₁=A and X₂=A; X₁=A and X₂=G; X₁=A and X₂=F; X₁=A and X₂=Q; X₁=G and X₂=A; X₁=G and X₂=G; X₁=G and X₂=F; X₁=G and X₂=Q; X₁=F and X₂=A; X₁=F and X₂=G; X₁=F and X₂=F; X₁=F and X₂=Q; X₁=Q and X₂=A; X₁=Q and X₂=G; X₁=Q and X₂=F; or X₁=Q and X₂=Q.

18. The recombinant retrovirus of claim 6, wherein the one or more morbillivirus envelope glycoproteins are measles virus F (MV-F) and measles virus H (MV-H).

19. The recombinant retrovirus of claim 18, wherein the MV-H protein comprises one or more amino acid substitutions at Y481, R533, S548, and F549.

20. The recombinant retrovirus of claim 18 or claim 19, wherein the MV-H protein comprises one or more amino acid substitutions selected from the group consisting of: Y481A, R533A, S548L, and F549S.

21. The recombinant retrovirus of claim 6, wherein the one or more henipavirus envelope glycoproteins are nipah virus F (NiV-F) and nipah virus G (NiV-G).

22. The recombinant retrovirus of claim 21, wherein the NiV-G protein comprises one or more amino acid substitutions at E501, W504, Q530, and E533.

23. The recombinant retrovirus of claim 21 or claim 22, wherein the NiV-G protein comprises one or more amino acid substitutions at E501A, W504A, Q530A, and E533A.

24. The recombinant retrovirus of any one of claims 1 to 23, wherein the non-viral membrane-bound tropism polypeptide comprises an extracellular antigen targeting domain, a spacer polypeptide, and a transmembrane domain.

25. The recombinant retrovirus of claim 24, wherein the extracellular antigen targeting domain binds an antigen expressed on an immune effector cell.

26. The recombinant retrovirus of claim 24 or claim 25, wherein the extracellular antigen targeting domain binds an antigen expressed on an immune effector cell selected from the group consisting of: the alpha, beta, gamma, or delta chain of the T cell receptor, CD2, CD3δ, CD3ε CD3γ, CD4, CD8α, and CD8β.

27. The recombinant retrovirus of any one of claims 24 to 26, wherein the extracellular antigen targeting domain comprises an antibody or antigen binding fragment thereof that binds an antigen expressed on the immune effector cell.

28. The recombinant retrovirus of any one of claims 24 to 27, wherein the extracellular antigen targeting domain comprises an anti-CD3 antibody or antigen binding fragment selected from the group consisting of OKT3, UCHT1, YTH12.5, TR66, and variants thereof, e.g., teplizumab, and antibodies and antigen binding fragments that have at least 80%, at least 85%, at least 90%, at least 95%, at least 99%, or 100% identity thereto.

29. The recombinant retrovirus of any one of claims 1 to 28, wherein the promoter is an inducible promoter or an MNDU3 promoter.

30. The recombinant retrovirus of any one of claims 1 to 29, wherein the polynucleotide encoding the chimeric antigen receptor is 5′ to the polynucleotide encoding the modular cell surface receptor.

31. The recombinant retrovirus of any one of claims 1 to 29, wherein the polynucleotide encoding the chimeric antigen receptor is 3′ to the polynucleotide encoding the modular cell surface receptor.

32. The recombinant retrovirus of any one of claims 1 to 31, wherein the modular cell surface receptor is a chemically inducible cell surface receptor.

33. The recombinant retrovirus of any one of claims 1 to 32, wherein the multipartite cell-surface receptor is a rapamycin-activated cell-surface receptor.

34. The recombinant retrovirus of any one of claims 1 to 33, wherein the modular cell surface receptor comprises a first T cell activating protein and a second T cell activating protein.

35. The recombinant retrovirus of claim 34, wherein (a) the first T cell activating protein comprises an FKBP-rapamycin complex binding protein (FRB) domain or a functional variant thereof and the second T cell activating protein comprises a FK506 binding protein (FKBP) domain or a functional variant thereof; or (b) the first T cell activating protein comprises the FKBP domain or a functional variant thereof and the second T cell activating protein comprises the FRB domain or a functional variant thereof.

36. The recombinant retrovirus of claim 34 or claim 35, wherein (a) the first T cell activating protein comprises an IL-2Rβ transmembrane and intracellular signaling domain and the second T cell activating protein comprises an IL-2Rγ transmembrane and intracellular signaling domain; or (b) the first T cell activating protein comprises IL-2Rγ transmembrane and intracellular signaling domain and the second T cell activating protein comprises an IL-2Rβ transmembrane and intracellular signaling domain.

37. The recombinant retrovirus of any one of claims 1 to 36, wherein the recombinant retroviral vector comprises a polynucleotide encodes a polypeptide that confers resistance to an immunosuppressive agent.

38. The recombinant retrovirus of claim 37, wherein the polypeptide that confers resistance to an immunosuppressive agent binds rapamycin, wherein optionally, the polypeptide comprises an FRB domain or a functional variant thereof.

39. The recombinant retrovirus of any one of claims 1 to 38, wherein the anti-CD19 chimeric antigen receptor comprises an antigen binding domain comprising an amino acid sequence set forth in SEQ ID NO: 93 or an antigen binding domain having at least 95% identity thereto.

40. The recombinant retrovirus of any one of claims 1 to 39, wherein the recombinant retroviral vector comprises a polynucleotide encoding a dominant negative TGFβ receptor or a TGFβ signal convertor.

41. The recombinant retrovirus of any one of claims 1 to 40, wherein expression of the chimeric antigen receptor is modulated by an FRB-degron fusion polypeptide and wherein suppression of the FRB-degron fusion polypeptide is chemically inducible by a ligand, optionally, wherein the ligand is rapamycin or a rapalog.

42. The recombinant retrovirus of any one of claims 1 to 41, wherein the viral envelope further comprises one or more transduction enhancers, wherein the transduction enhancer is selected from the group consisting of a T cell activation receptor, a NK-cell activation receptor, and a co-stimulatory molecule.

43. The system of claim 42, wherein the one or more transduction enhancers comprise one or more of CD80, CD86, CD137L, OX40L, and ICOSL.

44. A pharmaceutical composition comprising the recombinant retrovirus of any one of claims 1 to 43 and a pharmaceutically acceptable diluent.

45. A kit comprising the pharmaceutical composition of claim 44 and optionally a composition comprising a ligand, optionally wherein the ligand is rapamycin.

46. A method of treating a disease or disorder in a subject in need thereof, comprising administering to a subject, a therapeutically effective amount of the recombinant retrovirus of any one of claims 1 to 43 or the pharmaceutical composition of claim 44.

47. A method of treating a disease or disorder associated with malignant CD19⁺ or BCMA⁺ B cells comprising administering to a subject, the recombinant retrovirus of any one of claims 1 to 43 or the pharmaceutical composition of claim 44.

48. The method of claim 46 or claim 47, wherein the recombinant retrovirus is administered by intraperitoneal, subcutaneous, or intranodal injection.

49. The method of claim 48, wherein the recombinant retrovirus is administered by intranodal injection, via inguinal lymph node.

50. The method of any one of claim 46 or 49, where the disease or disorder comprises B cell malignancy, relapsed/refractory CD19-expressing malignancy, diffuse large B cell lymphoma (DLBCL), Burkitt's type large B cell lymphoma (B-LBL), follicular lymphoma (FL), chronic lymphocytic leukemia (CLL), acute lymphocytic leukemia (ALL), mantle cell lymphoma (MCL), hematological malignancy, colon cancer, lung cancer, liver cancer, breast cancer, renal cancer, prostate cancer, ovarian cancer, skin cancer, melanoma, bone cancer, brain cancer, squamous cell carcinoma, leukemia, myeloma, B cell lymphoma, kidney cancer, uterine cancer, adenocarcinoma, pancreatic cancer, chronic myelogenous leukemia, glioblastoma, neuroblastoma, medulloblastoma, sarcoma, and any combination thereof.

51. The method of any one of claims 46 to 50, where the disease or disorder comprises diffuse large B cell lymphoma (DLBCL).

52. The method of any one of claims 46 to 51, wherein at least 2 million, at least 4 million, at least 6 million, at least 8 million or at least 10 million or more transducing units of recombinant retrovirus are administered to the subject.

53. A method of transducing an immune effector cell in vivo, comprising administering to a subject, the recombinant retrovirus of any one of claims 1 to 43 or the pharmaceutical composition of claim 44.

54. A method of making a recombinant retrovirus comprising: a) transfecting the host cell with one or more polynucleotides that express retroviral gag-pol, one or more mutated viral envelope glycoproteins that retain fusogenic activity and lack cognate receptor binding activity, a non-viral membrane-bound tropism polypeptide, and optionally rev and a transfer vector encoding the recombinant retroviral vector of any of the preceding claims; and b) culturing the transduced cell for about 1 to 3 days to produce the recombinant retrovirus.
